(19) United States
(12) Patent Application Publication
(10) Pub. No.: US 2003/0044400 A1
(43) Pub. Date: Mar. 6, 2003

(54) Composition and method for treating cells

(76) Inventor: Jenny Yu, Durham, NC (US)

Correspondence Address: Colin P. Abrahams, Suite 400, 5850 Canoga Avenue, Woodland Hills, CA, 91367, US

(21) Appl. No.: 10/153,330

(22) Filed: May 21, 2002

Related U.S. Application Data
(60) Provisional application No. 60/292,574, filed on May 22, 2001.

(51) Int. Cl. A61K 38/16; A61K 38/43

(52) U.S. Cl. 424/94.1; 514/8

(57) ABSTRACT

The invention relates to a method and composition for a selective apoptosis factor that will specifically kill cancer and other target cells but not normal cells. The method is for use in the cancer therapy, for anti-aging, bone disease as well as other applications in human or animals. This invention is focused on the alpha 2-HS glycoproteins (AHSG) and their related clones.

### CROSS REFERENCE TO RELATED APPLICATIONS

This application claims the benefit of U.S. Provisional Application No. 60/292,574 filed May 22, 2001, which is incorporated herein in its entirety.

### FIELD AND BACKGROUND OF THE INVENTION

One aspect of the invention relates to a selective apoptosis factor that will specifically kill cancer cells but not the normal cells and is for use in the cancer therapy for human or animals. This invention is focused on Fetuin's human homologue, alpha 2-HS glycoproteins (AHSG) and their related clones. An important aspect is that the active domain of the drug effect is conserved between Fetuin and AHSG. However, due to immunogenecity, AHSG is a better form of drug than Fetuin. Recent research indicates that Fetuin is really a regulatory factor of cell proliferation and apoptosis. Fetuin's expression appears early during embryo development especially in the neuron crest, cerebrospinal fluid and limb buds. The biological function of Fetuin is quantitatively and qualitatively regulated throughout embryogenesis, the growing stage and aging. The apoptotic function of Fetuin beside pattern formation is mainly for cell regeneration at the growing stage and it is responsible for the tumorgenesis and cell senescence during aging due to the defective deletion of Fetuin molecule by telomerase activity. Many malignant patients have been found to have either a reduced level or an absence of Fetuin. Since hormone can activate telomerase, the status of Fetuin's function is strictly developmentally regulated. The apoptotic activity is regulated by the glycosylation modification of Fetuin molecules. With these characteristics, there can be applied therapy in anti-cancer, anti-aging and neuro-degenerated diseases or neurologic disorders in selective cell population and not affecting the normal cell population. The essence of Fetuin treatment is the specific high binding affinity of Fetuin to transforming growth factor-beta and a different affinity to other growth factors and cytokines. It creates an advantageous selective drug effect that does not present adverse reaction. Success has been achieved in preclinical tests in two rodents and one non-rodent species in hematological analysis, clinical chemistry, histopathological evaluation and immuno-suppression for safety evaluation. This application is primarily focused on claim of alpha 2-HS glycoprotein and its related clones in therapy of anti-cancer, anti-aging and neuro-degenerated diseases or neurologic disorders.

The invention also relates to the isoforms of AHSG. In one embodiment, there is a particular isoform which is transactivated to the tumor suppressor of p53. AHSG, also known as p63, has been claimed as a homologue of p53 due to the transactivation activity of isoform TAp63. However, Delta63 is not transactivated and not linked to glycosylation enzymes such as Glu. Synthetase does not affect apoptotic activity, since mature AHSG is deficient in expressing the modification enzymes in linking biology and loses the apoptosis-inducing activity. This explains the degeneration of cells and the turning on of tumorgenesis by losing the antagonized domain of AHSG. It also explains the specificity of AHSG between tumor cells and normal cells. Importantly, the hormone-binding domain of AHSG is divergent between bovine and human. For example, Fetuin's Mullerian Inhibiting Substance (MIS) homology domain (antagonized active domain) only overlaps 10 amino acids over three regions which contain a total of about 0.58 amino acids within AHSG molecules. The percentage of Homology (˜17%) is too low to postulate the same effect of Fetuin and AHSG.

This invention improves upon earlier uses of fetuin, disclosed, for example, in U.S. Pat. No. 5,994,298 which is incorporated herein by reference in its entirety.

### SUMMARY OF THE INVENTION

Recent works in various isoforms of p63 indicate that DeltaNp63 is the truncated form, and p63gamma (p51A) and p63alpha (p51B) are the full length. There is one particular isoform, TAp63 which can transactivate p53, tumor suppressing gene. Within the cell population, there are differences in the percentage of distribution of various isotypes. In some cancer cases, one form is much higher than others or vice versa. Auto-immunity characteristics within p63 gene is the part that is related to steroid hormone and divergent in individuals at C-terminal end. Most importantly, significant mutation usually falls into thr region of amino acids 151-170, which is the DNA-binding domain of p63 (hot spot) and is responsible for the Chronic Myelogenous Leukemia. At the C-terminal end of p63 about 50 amino acids are the domain and if deleted will affect the prostate development which is linked to Mullerian-Inhibiting Substance (MIS). Depending on the activity of Telomerase, aging syndrome and tumorgenesis usually are related to the deletion of p63 and reduced expression of p63 due to cell functional degeneration and play a role in replicative senescence.

AHSG is related to cell adhesion of lymphocyte and thymocyte of immune system responding to environmental factors and induced by cytokines such as IL-4. Developmentally speaking, its expression was initiated at the point that differentiation started at ectoderm, mesoderm and endoderm and modulated by hormone secretion as a regulatory factor. Terminal differentiation achieved the bone development of skeleton system. How a molecule plays such a diverse function of regulation relies on the fact that AHSG plays a role as reporter gene to bind and initiate the transcriptional events of associated functions modulated by hormones. Once the reporter gene was turned on, cells expressed the whole set of proteins that are linked in biological functions shown in differential display study. When regulatory factor expression is abnormal, it will either turn on tumorgenesis or cell senescence (programmed cell death). Preliminary data indicates that Fetuin, a bovine homologue of AHSG, can selectively induce apoptosis in tumor cell lines but not affect the normal cell lines. However, if you examine the binding domain of AHSG molecule itself, the homology only exists in TGF-beta binding domain and diversity at C-terminal end, which is linked to the antagonized function of AHSG as steroid and thyroid hormone initiators. This patent application therefore realtes to therapeutic usage of AHSG clones in human, which has different binding domain of hormone such as Mullerian-Inhibiting Substance (MIS) as bovine. It affects the androgen, estrogen and progesterone biosynthesis in cytochrome p450 oxidative enzyme series. In addition to the apoptosis-inducing activity, it can induce apoptosis of neuroblastoma and at certain concentrations, it can induce neurite outgrowth of PC-12 cells. The function of apoptotic activity can be controlled by the glycosylation modification of AHSG, therefore, an isoform of AHSG that is linked to Glucose synthetase is related to insulin and carbohydrate metabolism for protein modification in pancrease system and liver regeneration. There is another isoform, TAp63, that can transactivate the tumor suppresser gene, p53 and acts as an auto-immune agent for cancer. Preliminary data in animal tests also indicated that the selectivity of apoptosis activity of p63 can increase the life-span of leukemia-bearing mice and shrink the prostate tumors without presenting any detrimental side-effect as modern chemotherapy. This invention is therefore for the usage of AHSG related clones in therapy of cancer, aging-related degenerated diseases, AIDS, bone diseases and neuro-disorders in human.

Recent evidence indicated that AHSG is really a regulatory factor of cell proliferation, growth arrest and apoptosis. AHSG's expression appears early during embryo development especially in neuron crest, cerebrospinal fluid and limb buds. The biological function of AHSG is quantitatively and qualitatively regulated throughout embryogenesis, growing stage and aging. The apoptotic function of AHSG beside pattern formation is mainly for cell regeneration at the growing stage and responsible for the tumorgenesis and cell senescence during aging due to the defective deletion of AHSG molecule by telomerase activity. Many malignant patients were found to have either reduced level or an absence of AHSG. What is interesting is that in phase I and II Uterus cancer patients' specimen, AHSG gene is still intact, however, in phase III and IV Ovary and Cervics cancer patients' specimen, AHSG gene is deleted partially or completely, which is linked to Telomerase RT mRNA amplification. Since hormone can activate telomerase, the status of AHSG's function is strictly developmentally regulated. The apoptotic activity is regulated by the glycosylation modification of AHSG molecules. With these characteristics, we can apply therapy in anti-cancer, anti-aging, AIDS, bone and Neuro-degenerated diseases or neurologic disorders in selective cell population and not affecting the normal cell population. There are various isoforms of AHSG known as p63 alpha, p63 gamma, p63 beta and DeltaN p63 (truncated p63) existing in different percentage distribution within the cell population. In some cancer cases, certain forms of p63 are present much higher (TAp63) or absent completely (DeltaNp63) in the case of bladder cancer. The essence of a specific isoform of AHSG, TAp63, can transactivate the tumorsuppresser gene, p53, which affects the cell cycle regulation and turns on the apoptotic mechanism specifically in tumor cell population. It creates a wonderful selective drug effect that does not present any adverse reaction.

AHSG gene contain many domains with functional role. Amino acid 104-21 is very conserved and can bind TGF-beta with high affinity. It also contains DNA binding domain in amino acid 151-170. Many malignant patients' serum were found absent of this protein. Most importantly, at C-terminal end about 50 amino acid is the Mullerian inhibiting Substance homology domain that is served as steroid hormone and thyroidogenic factor initiator. It affects androgen synthetase mRNA expression and deletion of this domain will present anti-proliferative activity on breast cancer due to estrogen. It contains IL-4 binding domain to antagonize cytokines. Its expression is down-regulated by growth factors and cytokines indicated that it plays a regulatory role by high binding affinity. The specific clones are claimed to apply in therapy of various diseases as follows:

(1) The isoform linked to Myo D expression is responsible for both myogenesis and neurite development in connection of brain coordination and apoptosis of neuron in specific area will lead to paralysis and other neuro-disorders.

(2) The isoform of TAp63 will transactivate tumor suppressor gene, p53, and is linked to auto-immunity of bladder cancer and create a specific apoptosis-inducing activity only in tumor cell population.

(3) The isoform of p63 in chromosome 3 which is linked to Telomerase activity in woman cancer during aging and prostate cancer is linked to the deletion of MIS domain.

(4) The isoform of AHSG included the steroid and thyroid hormone initiator domain and linked to increase the immunity of AIDS patients.

(5) The isoform of AHSG is linked to Bone Morphogenic Protein-binding and Bone Resorption Protein in SMAD pathway and is responsible for bone mineralization and osteogenesis. The infection of osteoblast cells caused Arthritis and osteoporosis.

(6) Pancreatitis is linked to the isofrom of p63 connected to glucose synthetase and insulin. Liver Cirrhosis and Hepatitis is linked to the phosphorylation of cyclins and alkaline phosphatase. Retinoblastonia is linked to p34 phosphorylation. Chronic Myclogenous Leukemia is linked to mutation at amino acid 151-170 for DNA-binding domain. Parkinson disease is linked to translocation of 3q27-29, which is the AHSG gene domain, to chromosome 19 and affects the transcription of related protein expression. EEC syndrome is the autonomous dominant disorder characterized by ectodermal dysplasia and facial clefts which is due to mutation of DNA-binding domain of p63 that affects the initiation of transcription. All these linking biologies can be shown in differential display study about the related enzymes linked to its specific function in various organs. We can address specifically in individuals deficiency related to the regulatory role of AHSG in various clones by binding affinity.

AHSG plays a fundamental role in regulating the whole function of our body by serving as a hormone reporter gene to turn on or off many related transcriptional events as antagonized, modulating factor for not only growth factors but cytokines as well. Many therapies in the drug industry target on one mechanism whereas the invention is focused on naturally how growth regulation is controlled and why it will turn on tumorgenesis and aging-related diseases and how can we fixed that change during aging and cure cancer. When a protein plays a fundamental role in our body, it is the essential protein to compensate the genetic change of this protein toward aging and cancer shown in differential display study. The hormone-binding domain is divergent among species which is the theory of antagonization related to steroid hormone and cytokines, especially related to the immunity and used in immuno-deficient patients particularly. However, the TGF-binding domain is homologous enough among species. Therefore, depending on the genetic alteration due to virus or bacterial infection in individual cases, it determines whether it can be applied or not.

Different individuals have different changes genetically during virus infection altering the protein expression. Sometimes, it is detrimental because it is the functional domain for protein activity. Sometimes, it will not affect at all if mutation is not in region of important function. In general, we can provide the clones of functional protein to compensate the deterioration of aging or antagonized tumorgenesis.

AHSG itself is a carrier protein since it binds to many functional growth factors and cytokines extracellularly. It can be administered through i.v. or i.p. It may not be as other drugs that need a carrier to introduce to the target place if it is not hydrophobic enough to pass through lipid bilayer and get into the cells. AHSG itself is the signal regulatory complex and served as a growth factor receptor in some cases.

AHSG should preferably administer as itself, because it is a natural protein in our body, not as herbal extract or chemicals.

In the case of cancer, if it is phase I and II, it is locally in certain tissues, we can make clones targeted on certain tissue specific hormone-binding domain. If it is phase III and IV, it is metastasized to various tissues, it can be introduced as a full length of protein that targets on many hormones for various tissues. In the case of aging, it can depend on the degenerated tissues or the tissue that is deficient to make the full length of AHSG or express inefficiently in quantity. In the HI V-infected individual, it has to enhance on the steroid hormone initiator domain for auto-immunity. In the case of prostate, it is the Mullerian-Inhibiting Substance domain that should be targeted in clones. In the case of bone diseases, it is the BMP-binding domain or glycosylation modification that control the apoptotic activity of this protein such as sialic acid mosiety. Completely understanding the individual symptoms and alteration of AHSG expression not only quantitatively but qualitatively will faciliate the therapy in accuracy.

### I. Neurological Assessment and Applications

Communication between cells is mediated by the endocrine, nervous and immune system, which constitute an interlocking network. The network is controlled by a transcriptional regulatory factor such as AHSG which is believed to intercalate in selective evolution through virus. The characteristics of this essential factor contain a DNA-binding domain, hormone-binding domain and a N-terminal variable or immunodominant domain. The interlocking nature of this relationship is mostly apparent in the hypothalamus, where a neuro-endocrine-immunologic system evolved to integrate and coordinate the metabolic activities of higher organisms. AHSG is a molecule playing a role in this interlocking network. The expression of AHSG is downregulated by many growth factors and cytokines. The significance of this study can apply in many neurological disorders due to endocrine secretion, nervous system communication, immune response during the onset of bacterial pathogenesis and virus infection, chemical impulse due to environmental factors, accidental injury or hypoxia damage and neuro-degenerated diseases during aging.

The receptor of AHSG is only found in the cells transformed by Epstain-Barr virus but not in normal cells.(5) The Epstain-Barr virus glycoprotein binds to the B lymphocyte complement receptor CD21. Other viruses encode membrane proteins with enzymatic activity for receptor destruction. Influenza virus (a respiratory infection virus), for example, encodes a glycoprotein with neuramindase activity, which destroys sialic acid on the infected cell's plasma membrane. The onset of disease has many stages in the development. The severity is dependent on how our defense system responds to the foreign intruders. The interaction of virus with its receptor usually induces receptor aggregation at the site of viral adsorption. The aggregated receptor is internalized through an endocytic process that involves clathrin-coated pits. Influenza hemagglutinin mediates adsorption, receptor aggregation and endocytosis. Influenza virus is further subtyped on the basis of the surface hemagglutinin (H) and neuramindase (N) antigen. The hemagglutinin is the site by which the virus binds to the receptors, whereas neuramindase degrades the receptor and probably plays a role in the release of the virus from infected cells after replication has taken place. H1N1 virus circulated from 1918 to 1956; thus individuals born prior to 1957 would be expected to have some degree of immunity to H1N1 virus.

The study of AHSG involves the evaluation of neurological responses based on the Electroencephalogram (EEG), Echo-planar Magnetic Resonance Imaging and a genomic analysis. The EEG records the electrical activity in the brain from the electrode placed on the scalp. The findings depend on the patient's age and level of arousal. The rhythmic activity represents the postsynaptic potentials of vertically oriented pyramidal cells of the cerebral cortex and is characterized by its frequency. The Echo-planar Magnetic Resonance Angiography gives an image of magnetic resonance interaction between protons in biologic tissues and blood flow, a static and alternating magnetic field and energy in the forms of radio frequency waves of specific frequency. It can detect the ischemic injury and is used in somatosensory localization that alters the balance of oxyhemoglobin and deoxyhemoglobin. The genomic analysis can precisely detect the molecular disorder due to genetic alteration, especially relevant to the deletion of AHSG during cell senescence such as deficiency in eye sight, hearing ability, cardiac failure and memory loss.

The assessment of Fetuin treatment in terms of hematological parameter, clinical chemistry and histopathological analysis in mice, rat and rabbits has been carried out. Fetuin treatment also did not present any immunosuppression activity. Literature searches show many biological links in signal transducer and transcription activator pathways with characteristics in growth hormone and cytokine family. Receptor of this family does not have kinase domain, but ligand binding results in the rapid phosphorylation of tyrosine residues on the receptor itself and on cellular proteins. AHSG is the extracellular regulatory factor to stimulate or antagonize cell proliferation and apoptosis. Genomic study of AHSG also found a direct link in cell proliferation and apoptosis. In addition, it can be used in therapy of neuroblastoma and neuro-degenerated diseases.

Results of Preliminary Data

1. Isolation of Nerve Survival Factor (NSF) and Identified as Fetuin

The XC cells were grown in 10% FBS and MEM media to confluence and changed to serum-deprived MEM media for 4 days. The conditioned media were collected and partially purified to homogenous in anion-exchange chromatography and size exclusion chromatography on HPLC. The fraction was found to contain an activity preventing cell death in PC-12 cells. PC-12 cell is a neuron cell line that usually dies when grown in the absence of fetal bovine serum The cell death of PC-12 cells is characterized as apoptosis. It was found that in the presence of the concentrated conditioned medium of XC cells, the cell death of PC-12 cells caused by serum starvation is inhibited. All of the PC-12 cells are found to be dead. However, in the presence of 0% fetal bovine serum and concentrated conditioned medium of XC cells, most of the cells are still alive. A protein with apparent molecular weight of 60 Kd on the size exclusion chromatography was observed. This apoptosis-inhibiting protein was designated as Nerve Survival Factor (NSF). The purified NSF was run in 4-20% Tris-Gly gel on SDS-PAGE and transferred to PMVF membrane. The NSF was identified by N-terminal Amino Acid Analyzer as Fetuin.

2. Low Concentration of Fetuin Stimulate Neurite Outgrowth of PC-12 Cells.

Fetuin was purified with modified Spiro's Method from fetal bovine serum. Three hundred milliliters of fetal bovine serum added 180 ml of 0.1 M Zinc Acetate, 180 ml of 95% cold Ethanol and 240 ml distilled water and adjusted pH to 6.4 with 1 M NH₄OH—NH₄Cl Buffer, pH 10.4 in 19% Ethanol. The mixture was stored at −5° C. for 16 hr and centrifuged at 8,000 rpm for 15 min. The 750 ml of supernatant added 17.3 ml of 1M Barium Acetate and 33 ml of 95% cold Ethanol and stored at −5° C. for 2 hr. The mixture was centrifuged at 8,000 rpm for 15 min. The 760 ml of supernatant added 225 ml cold Ethanol and stored at −10° C. for 16 hr. The mixture was centrifuged at 8,000 rpm for 15 min. and Fetuin pellets were dissolved in 60 ml pyrogen free sterile saline solution. Fetuin was dialyzed against distilled water for 1-2 days with 3 times of change of water. The protein solution was sterilized by filtration with 0.2 um membrane. The concentration of protein was quantitated by Bio-Rad DC Protein Assay. The purity of the protein was analyzed by SDS-PAGE and N-terminal amino acid sequence determined as 99.5% pure.

PC-12 cells were seeded in 10% heat-inactivated horse serum, 5% fetal bovine serum in RPMI 1640 medium for 1-2 days. The media were changed to 0.5% serum+RPMI overnight and treated with 5uM Fetuin. The neurite outgrowth was observed in many of the cells over 2-3 days.

3. High Concentration of Fetuin Induce Apoptosis of Neuroblastoma Cell Lines

PC-12 cells were seeded in 10% heat-inactivated horse serum+5% FBS+RPMI 1640 media for 1-2 days and treated with 5 uM Fetuin. The apoptosis-inducing activity was observed even in the presence of serum. Fetuin was found to be able to bind transforming growth factor-beta (TGF) with high affinity. The physiological concentration of transforming growth factor was 1000-fold higher than needed and the association constant (Ka) and deassociation constant (Kd) indicated that the on and off rate is sufficient to activate or inhibit the biological function of TGF depending on the concentration of a regulatory factor, Fetuin, that synergizes or antagonizes the effect of TGF. The apoptosis-inducing activity of Fetuin at high concentration can be applied in therapy of neuroblastoma.

4. Therapy of Neurological Disorders

The central nervous system (CNS) is the core of all the communication between brain and different organs. Electroencephalography provided the first indication of location of impairment. Detailed examination continued with a specific diagnosis of the void region. Many imaging methods can be used for assessment of treatment. Norrie disease is a neurologic disorder with clinical symptom of retinal malformation, hearing loss and mental retardation. This is an X-linked recessive mutation disease at Xp11.4. The sequence comparison and modeling suggested similarity to TGF-beta family/cysteine-knot motif growth factor. Fetuin can bind TGF-beta and bone morphogenic protein (BMP). Fetuin has been claimed to play a role in programmed cell death of osteoblasts during osteogenesis. It also plays a role in programmed cell death of pattern formation during embryogenesis. Another X-linked recessive neurologic disease, hydrocephalus, has a mutation of L1 gene, located at Xq28. L1 gene product is a multifunctional/multidomain cell adhesion molecule. The expression of L1 is regulated by thyroid hormone. Thyroid hormone is essential for brain maturation, regulating neuronal differentiation and migration, myelination and synaptogenesis. Mutation of the adhesion molecule L1 causes severe neurological abnormalities in humans. Fetuin is a fetal protein with characteristics very similar to L1 gene, containing very conservative multifunctional/multidomain with a variable N-terminal immunodominant domain. It is expressed highly during fetal development. Spiro first purified this protein and claimed its function for cell adhesion in 1944; however, its function has never been clear up to today. Evidently it is an amazing integration in evolution due to virus infection. An electrophysiological approach provides an excellent tool to facilitate the evaluation of Fetuin in therapy of neurologic disorders in an efficient and sensitive way. Many neuro-degenerated diseases are derived from aging. Aging has been linked to an enzyme, telomerase, that will delete Fetuin, a regulatory factor of cell proliferation and apoptosis controlling the development of embryogenesis, growing stage through hormone secretion and senescence due to telomerase activity. The essence of Fetuin treatment is specifically targeted on the onset of diseases and selective in only infected cells due to the fact that receptors of Fetuin are only found in transformed cells. The natural specificity created a perfect therapy for whole body evaluation of tumorgenesis and senescence, especially in neuro-degenerated diseases.

### EXPERIMENTAL DESIGN AND METHODS

1. Comparison of Mental Stimulation and Fetuin Therapy in Related Neuro-Deficiency.

A. Mental Status Examination:

These tests are designed to evaluate attention, orientation, memory, insight, judgement and grasp of general information. A series of numbers can be recited and the patient asked to respond if a specific item recurs (attention). Retentive memory and immediate recall can be tested by determining the number of digits that can be repeated in sequence. Recent memory is evaluated by testing recall of a series of objects after defined times. More remote memory is evaluated by assessment of a patient's ability to provide a coherent chronologic history of his or her illness or personal life event. Recall of a major historical event or current events can be used to assess the fund of general knowledge. Interpretation of proverbs can provide information relative to judgement and capacity to reason in an abstract manner. Specific higher cortical functions can be tested when clinical circumstances suggest that deficits may be present.

B. Coordination:

The patient is asked to touch his or her index finger repetitively to the nose and then to the examiner's outstretched finger. Observation of gait is an essential component of the neurologic examination. Normal gait requires multiple systems such as power, sensation, coordination and praxis functioning in a coordinate fashion.

C. Electrophysiologic Study of Central and Peripheral Nervous System:

Electroencephalography

Normal EEG is recorded as an 8-13 Hz alpha rhythm posteriorly intermixed with a variable amount of generalized faster (beta) activity. During drowsiness, the alpha rhythm is attenuated; with light sleep, slower activity in the theta (4-7 Hz) and delta (<4 Hz) range become more conspicuous. For patients with seizure, the EEG have abnormal, repetitive rhythm activity with an abrupt onset and termination. For patients with coma, the EEG becomes slower as consciousness is depressed. It can also be used in diagnosis of certain neurological disorders such as patients with acute encephalopathy of herpes simplex encephalitis and periodic lateralized epileptiform discharge commonly found in acute hemispheric pathology such as hematoma, abscess or rapidly expanding tumor.

Magnetic Resonance Imaging (MRI)

With echo-planar MRI, fast gradients are switched on and off at high speeds to create the information used to form an image. The rate of return to equilibrium of perturbed protons is called the relaxation rate. The relaxation rate is different for normal and pathological tissues. The relaxation rate of hydrogen proton in a tissue is influenced by surrounding molecular environment and atomic neighbors. The T1 relaxation rate is the time for 63% of protons to return to their normal equilibrium state. The T2 relaxation rate is the time for 63% proton to become dephased owing to interactions among adjacent protons. The intensity of the signal and thus the image contrast can be modulated by altering certain parameters, such as the interval between Rf pulses (TR) and the time between the Rf pulse and the signal reception (TE). So-called T1-weighted (T1W) images are produced by keeping the TR and TE relatively short. T2-weighted (T2W) images are produced by using longer TR and TE times. Normal MRI scan of the brain in T2W image will give an image that gray matter is slightly higher in signal intensity than white matter due to more lipid (myelin) content. Cerebrospinal fluid (CSF) has a bright signal intensity owing to its free mobile water content. Moving protons in arterial structures demonstrates a signal void after ischemic injury of the brain. In T1 image, less contrast is visible between gray and white matter structures—white matter appear slightly higher in signal intensity than gray matter owing to a shorter T1 relaxation time. The image can identify the disconnection of brain and different functional regions.

Genomic Analysis

As NIH whole human genome project is accomplished, we can approach the genetic polymorphism for individual need. The feasibility of human linkage analysis was revolutionized by the demonstration of genetic variation in the size of fragments generated by digestion of normal human DNA with restriction endonucleases. These restriction fragment length polymorphisms (RFLP) are the consequence of the DNA sequence polymorphisms and are inherited according to Mendelian principles. Restriction enzyme digestion and southern blotting make it possible to utilize these polymorphisms as genetic markers for sites within the genome. If one of the base pairs in the recognition sequence for a restriction enzyme differ between individual copies of the genome or if there is a length variation in the DNA, there will be variation in the size of the DNA fragment generated by the restriction enzyme digestion. The advantage of genome analysis is targeting defects specifically in individual alteration. Two of neurological disorders in this approach were linked to an important region controlling the signal transduction complex for cell proliferation which leads to night blindness (3q 21-24) as well as retinal angioma, CNS hemangioblastoma, and pancreatic cysts (3p25-26). VHL 1 gene located at 3p25-26 encodes a tumor suppressor gene that plays a regulatory role in expression of VEGF.(20) Fetuin, at the other end of chromosome 3, plays a regulatory role in expression of TGF and other growth factors and cytokines. Deletion of both loci is cytogenetically linked to various sporadic tumor incidences. They both served as an endocrine tumor suppressor gene related to the neuron communication network. Deletion of Fetuin gene due to repression or amplification of telomerase activity will lead to growth out of control (tumorgenesis) or cell senescence.

### II. Anti-Cancer Therapy

Alpha 2-HS glycoprotein (AHSG), a Fetuin homologue, is an important plasma protein that is related to the pathogenesis of diseases and the immunity of our self-defense system. Its receptor was found on the lymphocytes transformed with Epstein-Barr virus but not the normal lymphocyte. The transforming phenotype related to the cell adhesion characteristics is due to the domain 3 of AHSG at RGD region (Arg-Gly-Asp) for glycosylation. The divergence of two forms (50 kDa and 60 kDa) of AHSG observed in the HepG2 and normal liver cells indicated that antibodies recognize the B chain only found in the HepG2 but not the normal cells. The glycosylation form of AHSG affects the apoptosis-inducing activity that causes transformed and normal cells to have different growth characteristics. Synthetic peptides with promoting activity of cell adhesion in heavy chains may be an effective reagent for wound healing in tissue injured by inflammation. Furthermore, the addition of a Fungi metabolite, brefeldin A, which will block the transport of protein from the endoplasmic reticulum to the Golgi complex, all AHSG synthesized remained only in 40 kDa form of AHSG (precursor form) and no AHSG was secreted into the culture medium. However, the pulse-chase labeling of AHSG after treatment with Endoglycosidase H in HepG2 cells indicated that the processing of 50 kDa to 60 kDa form occurred after the protein was transported to the late Golgi complex and only 50 kDa and 60 kDa forms can be secreted in the culture medium. The secretion and cell adhesion result in the metastasis of cancer. AHSG acts as an opsonin during bacterial phagocytosis by neutrophils and enhances macrophage phagocytic function. AHSG can bind Epstein-Barr virus DNA and play a role in host defense mechanism against virus infection. Over expression of AHSG in the adipose cells inhibits both basal and insulin-stimulated phosphorylation of Elk-1, a transcription factor phosphorylated and activated by mitogen-activated protein kinase. AHSG in the media can be internalized effectively by adipose tissues. The mechanism of tumorgenesis, inflammation, pathogenesis and adipogenesis all relate to the glycosylation form of a growth regulatory factor that affect the cell adhesion, apoptosis activity and metastasis. The fact that autoimmunity of Endometriosis women have elevated levels of AHSG and antibodies of AHSG inhibit the sperm mobility suggested AHSG plays a role in infertility of Endometriosis women. Therefore, it is important to analyze the carbohydrate property of AHSG in diagnosis, prevention and therapy of cancer or aging-related diseases.

Fetuin, the human homologue of AHSG, was found to induce apoptosis in cancer cell lines in 30 min—2 hr but not the normal cell lines in 2-3 days. Since embryogenic cell lines have many characteristics similar to cancer cell lines and only fetal Fetuin can induce apoptosis but not the mature Fetuin, AHSG behaves like a carcinoembryogenic antigen. The difference between fetal Fetuin and mature Fetuin is the carbohydrate property. Fetal Fetuin is sialic acid-rich (8.7%) and mature Fetuin is sialic acid-poor (0.2%). The carbohydrate property of AHSG determine a tertiary structure that will expose the binding site to transforming growth factor (TGF) and quenching the effect of TGF and inducing apoptosis. The purpose of apoptosis is for pattern formation during embryogenesis and cell regeneration during growing stage. At a mature stage, the ability of cell regeneration is gradually abolished by less apoptosis to revive the cell population and finally reach the senescence stage. The molecule control the development of growth is a regulatory factor, AHSG. Since premature aging is linked to an enzyme, 3′-5′ exonuclease and the AHSG gene is located at the end of chromosome 3q27, the deletion of such an important gene during cell division affects the whole process of growth. Many elderlies were found either to have a reduced level of Fetuin, defective Fetuin (in other words, deleted Fetuin) or an absence of Fetuin, which lead to tumorgenesis or out of growth control. Therefore, tumorgenesis is a process of senescence where AHSG is completely absent to regulate growth. Malignant patients lose weight because they can not sustain the aggressive growth of tumor cell population and normal cell population reaches a senescence stage.

The onset of tumorgenesis is earlier in some cases due to the integration of a virus transcription promoter that causes over expression of genes, named oncogenes. Oncogenes were found to be the genes related to growth factors, such as erb for EGF. Since growth factors and cytokines will down-regulate the expression of AHSG, the supplement of AHSG can quench the growth stimulation effect of growth factors that is over expressed due to virus infection. Another reason would be the mutation caused by environmental factors such as chemicals, naturally occurring carcinogens in foods and radiation or oxidation of oxygen radical. A single gene mutation or substitution will affect the genetic code of protein expression. If the mutation occurred in an important domain that affects the biological function of that protein, it is a detrimental mutation. It is fortunate that the biological function domain of AHSG is very conserved among species; however, the domain 3 or chain B is very divergent. It is a domain that is only found in virus infected cell lines and have homology to Mullerian Inhibiting Substance (Inhibin). During the development, an onset of virus infection integrates a segment of chromosome in lymphocytes that will build some antibody in our self-defense system for natural selection. The normal cells not infected by virus only contain A chain. PDGF research indicates that BB dimer will inhibit AA dimer induced calcium impulse and mitogenesis. B chain antagonizes A chain's effect. Therefore, the heterodimer has an inhibitory effect to homodimer.

Many enzymes are inducible by substrates. Neuramindase is an enzyme that will hydrolyze the terminal carbohydrate, sialic acid. The expression of neuramindase is related to the pathogenesis of bacterial infection and neoplasm. Neuramindase is a marker for tumor cells. The addition of Neuramindase will abolish the apoptosis-inducing activity of fetal Fetuin. Many malignant patients' serum were found to have higher amount of sialic acid and lower amount of AHSG. Women with endometriosis were found to have elevated AHSG and autoimmune system. AHSG has been claimed to be the inhibitory factor in follicular fluid for oocyte maturation and elevated AHSG will inhibit the sperm mobility and cause infertility.

AHSG has been linked to osteogenesis by binding to Bone Morphogenic Protein (BMP) during osteoblast mineralization. AHSG can be a calcium phosphate absorbent due to the negatively charged sialic acid. Studies on chondrocytes indicated that AHSG can stimulate Alkaline Phosphatase and Phospholipase A₂ and it is only observed in the growth zone of chondrocytes but not in the resting zone of chondrocytes. Patients with Rheumatoid Arthritis also have reduced levels of AHSG after inflammation. The mRNA transcript of AHSG in chondrocyte is sized differently from AHSG synthesized in liver, 2.2 kb and 1.6 kb respectively. The AHSG synthesized in liver is a phosphorylated form of AHSG and the circulating AHSG is a non-phosphorylated form. Since phosphorylation of tyrosine kinase activates the signal transduction of a proliferative signal which will induce the dimerization of growth factors and Phospholipase A_{,2} activity was increased during inflammation to release a second lipid messenger, arachidonic acid, activated due to GTP binding and transduced signal to raf. Raf further phosphorylated MAP Kinase and transduced to fos and myc nuclear factor. The whole cascade of signal transduction is turned on by phosphorylation and turned off by dephosphorylation. The cell cycle of mitosis is also regulated by phosphorylation of cyclin, p34. The upstream of this signal transduction cascade is the AHSG regulatory factor. In addition to T-cell mediated immune response, exosomes are small vesicles released by antigen-presenting cells and may have an immune-regulatory function in vivo. Exosomes originate from Major Histocompability Complex (MHC) class II and expressed B-cell marker, CD20 and complement inhibitory protein, CD59. A Zn-alpha2-glycoprotein with sequence homology to MHC class II was reported and related to the adhesion of platelet coagulation during inflammatory response. Taken together, AHSG and cystatin family, have a role in autoimmune system. AHSG-induced apoptosis is involved in MAPK-mediated growth arrest. Whether it accumulates cyclin D1, D3, E and A through NF-kappa B activation or not is unknown; however, AHSG 5′-flanking region contain several C/EBP and NF-1 binding site.(36)

The mechanism for this regulatory role is by binding to the growth factors and cytokines and control the availability of physiologically functional concentration of growth factors. AHSG is the key factor of the signal transduction pathway. It is the competitive phosphorylation between tyrosine kinase, GTP and GDP-ras or kinase and phosphatase to switch on and off in the cascade. Considering the speed of proliferation and apoptosis, AHSG is very potent and faster in controlling these biological functions than any other factors in the signal transduction cascade.

In addition to TGF and BMP, AHSG was also found to be able to bind sarconectin, hemonectin, lymphokine migration inhibitory factor, glycosylation inhibitory factor and macrophage migration inhibitory factor. It has been claimed to play a role as growth factor receptors. AHSG was reported to be able to bind HGF, PDGF and insulin. It plays a regulatory factor role in growth and apoptosis. With such a profound role in our body, it is an important carrier protein for many biological functions. Whether this molecule is deleted gradually during evolution, cell division or senescence is an important issue.

The direction of scientific research on glycosylation, protein processing at Golgi complex or mitochondria oxidation, is all aimed to the glycosylation of an important molecule, AHSG, that regulates the whole process of growth and apoptosis during development. The expression of AHSG is developmentally regulated. The biological function of AHSG is to control growth and apoptosis and is regulated by the glycosylation form of the molecule. Any defect related to gene amplification, gene transcription, protein processing and secretion of AHSG will be compensated for by supplementing AHSG. A differential display study in a Switzerland group indicated that Liver Cirrhosis patients expressed a defective AHSG, 41 kDa instead of 48 kDa precursor and the expression level is reduced to 45% of normal level. Since senescence has been linked to telomerase, which is an enzyme shorten the chromosome end and a premature aging syndrome protein has been demonstrated to be a 3′-5′ exonuclease, the enzyme delete an important gene, AHSG, that directly control cell growth and apoptosis.

Study of the 5′-flanking region of the AHSG gene indicated that element I has a C/EBP and NF-1 binding site, element II up regulate the expression of AHSG gene and element III is a negative silencer. The proximal promoter region about 300 nt is very conserved between species and beyond the proximal promoter region is divergent. Since interleukin-1 downregulates the expression of AHSG, it would be interesting to understand how these regions affect the expression of AHSG during inflammation and carcicogenesis. AHSG was found to be able to bind IL-4 and play a role as an opsonin for macrophage phagocytosis. AHSG functioned as an antagonist of TGF and cytokines and can be served as an anti-tumor immune antigen. Exogenous addition of AHSG is a novel approach for cancer therapy.

Fetal bovine fetuin prepared in a modified Spiro method was found to induce apoptosis in several cancer cell lines such as LNCaP and PC-3 (Prostate cancer), HL-60 (Leukemia), Calu-1 (Lung cancer), MCF-7 (Breast cancer), Colo-205 (Colon cancer) and HepG2 (Liver cancer) but not the normal cell lines such as CCD18Co (Normal colon), CCD39Lu and Wi-38 (Normal lung). The potency of apoptosis-inducing activity of Fetuin was compared to current market drugs such as Bleomycin, Doxorubicin, Taxol, 5-Fu, Mitomycin and Tamoxifen. Fetal Fetuin is superior in terms of as fast as 30 min to 2 hr., not to mention the specificity targeting only tumor cells. Fetal fetuin tested in a P388 Leukemia model with 5 consecutive injections was found to increase the survival rate of leukemia-bearing mice 141% compared to the control group. (8 out of 10 mice survive). In a placebo (saline) treatment, all 10 mice died. Fetal fetuin tested in a prostate tumor model can reduce the tumor size 100% in PC-3 induced tumor formation in mice compared to the control. (In a Placebo treatment, the tumor size was 2.34 g). The advantage of 5 consecutive injections was to keep the effective dose overtime during the treatment. When we use 2.5-5 fold higher dose that is effective in cancer-bearing mice to treat healthy mice and evaluated by hematological, clinical chemistry and histopathological analysis, it was found safe and no obvious adverse effect was observed. The glucose and cholesterol levels were a little higher in the highest dose but not statistically significant.

Fetal bovine Fetuin was suggested to play a role in osteogenesis in the mineralized tissues. The expression of fetuin in mineralized tissues is 30-100 fold higher than the normal tissues. Fetuin was also shown to be able to bind TGF-beta and BMP which linked to programmed cell death in osteoblast cells. Genomic studies linked Fetuin to play a role in growth arrest and apoptosis. The mechanism of biological function of Fetuin is to control the availability of growth factors and cytokines. Several cytokines down-regulate the expression of Fetuin. Decreased levels of human Fetuin have also been observed in patients with acute lymphocytic leukemia, chronic granulocyte and myelomonocyte leukemia, metastasized solid tumors, Hodgkin's lymphomas, multiple myeloma, acute alcoholic hepatitis, chronic active hepatitis, acute and chronic pancreatitis. The fact that tumor cells expressed different levels of transforming growth factor than the normal cells create a natural specificity for this novel approach of cancer therapy.

In conclusion, Apogen F (commercial name for Fetuin) is a regulatory factor that controls the growth arrest and apoptosis by the concentration of Fetuin. A high concentration of Fetuin will bind the growth factors to initiate a growth inhibition signal, inhibit MAP kinase phosphorylation and induce apoptosis. A low concentration of Fetuin will activate growth factor and induce cell proliferation. In other words, the variation of Fetuin concentration will enhance the metabolism and regeneration of a cell population. The fact that neuramindase is the marker of tumor cells and neuramindase abolishes the apoptosis-inducing activity of Fetuin leads to the outgrowth of tumor cells. The Fetuin gene is located at chromosome 3q27 and the telomerase gene is located at chromosome 3q26.3. The shortening of that region deletes the Fetuin gene and affects the expression of fetuin. Telomerase studies have been suggested to link to tumorgenesis. Contraindications exist to correlate the telomerase and tumorgenesis: (1) Telomerase RNA expression can be detected in the absence of enzyme activity. (2) The tumor incidences that related to telomerase and growth arrest are only connected to chromosome 3 but not the other chromosomes such as 7, 8, 11, 12, 20 and 21. (3) The expression of telomerase catalytic unit (hTERT) correlate to the tumor incidence only in stages I and II but not in stage III. Therefore, the biological phenomenon observed in the telomerase studies is actually the biological function of Fetuin. Many malignant patients were found to have reduced level of alpha 2-HS glycoprotein, Fetuin human homologue or an absence of alpha 2-HS glycoprotein. The supplement of Fetuin treatment will induce apoptosis or reduce the growth of tumor cell population and not affect the normal cell population or even stimulate the metabolism and regeneration of normal cell population. This novel approach has no adverse effect. Since the drug effect is extracellular, there is no delivery problem.

Experimental Design and Data

A. Bovine Fetuin Contains Activity Inducing Apoptosis in Tumor Cells

We have isolated an apoptosis factor from Fetal Bovine Serum and amino acid sequence analysis indicated that the 60 Kd protein on the SDS-PAGE is Fetuin. We purified the fetal bovine fetuin by the modified method of Spiro described as the following:

(I) Purification of Fetuin from Fetal Bovine Serum

Three hundreds milliliters of fetal bovine serum added 180 ml of 0.1 M Zinc Acetate, 180 ml of 95% cold Ethanol and 240 ml distilled water and adjusted pH to 6.4 with 1 M NH4OH—NH4Cl Buffer, pH 10.4 in 19% Ethanol. The mixture was stored at −50 C for 16 hr and centrifuged at 8,000 rpm for 15 min. The 750 ml of supernatant added 17.3 ml of 1M Barium Acetate and 33 ml of 95% cold Ethanol and stored at −50 C for 2 hr. The mixture was centrifuged at 8,000 rpm for 15 min. The 760 ml of supernatant added 225 ml cold Ethanol and stored at −100 C for 16 hr. The mixture was centrifuged at 8,000 rpm for 15 min. and Fetuin pellets were dissolved in 60 ml pyrogen free sterile water. Fetuin was dialyzed against distilled water for 1-2 days with 3 times of change of water. The protein solution was sterilized by filtration with 0.2 um membrane. The concentration of protein was determined by Bio-Rad DC Protein Assay. The purity of protein was analyzed by SDS-PAGE and amino acid sequence analysis indicated that 99.5% purity was achieved.

(II) Fetal Fetuin Induces Apoptosis in Tumor Cell Lines Without Affecting Normal Cell Lines

The purified fetuin was tested on various cancer cell lines. Fetal fetuin strongly induced apoptosis in certain cancer cell lines such as: LNCaP (Human prostate adenocarcinoma), PC-3 (Human metastatic prostate adenocarcinoma), HL-60 (Human promyelocyte leukemia), MCF-7 (Human Breast adenocarcinoma), Colo 205 (Human colon carcinoma), Calu-1 (Human lung carcinoma) and HepG2 (Human hepatoma). However, fetuin did not induce apoptosis in normal cell lines such as CCD18Co (Normal human colon fibroblast), CCD39Lu and WI-38 (Normal human lung fibroblast). The DNA condensation and breakage of nuclei which are the characteristics for apoptosis were observed by staining the cell with Hoechst dye at 0.1 ug/ml.

(III) Zinc is an Important Factor for Apoptotic Activity of Fetal Fetuin

An important observation made during this study is that Zinc seems to be a critical factor affecting fetuin's apoptosis-inducing activity. The commercial fetuin purchased from Sigma which is prepared by the Pederson Method did not show apoptotic activity. This may be due to the use of EDTA which deprives the metal ions bound to fetuin during the process of purification. The fetuin prepared with the modified Spiro method, however, induced apoptosis. Substitution of Zinc by Barium completely abolished the apoptosis-inducing activity of fetuin. The LD50 of Zinc-charged fetuin was determined in various cancer cell lines shown on Table 1.

Fetuin purified by modified Spiro method was further incubated with Zinc Acetate or Barium Acetate (0.25M) at room temperature for 1 hr. Free ions were removed by repetitive concentration against 20 volumes of PBS three times. Fetuin, Zn-charged fetuin and Ba-charged fetuin (5 uM) and PBS were then separately incubated with LNCaP cells for 4 hr. Percent of cells under apoptosis was determined by Hoechst staining and confirmed by MTS assay. Each data represents the average of two assays. Zn-charged or Ba-charged fetuin may contain free Zn or Ba (about 30 uM). However, all the cell lines are not affected by Zn or Ba alone at all concentrations tested (up to 66 uM).

TABLE 1
LD50 of Fetuin-induced Apoptosis in Various Cell Lines

| Cell Lines | LD50 | |
|---|---|---|
| LNCaP | 1 | uM |
| PC-3 | 1 | uM |
| Colo 205 | 5 | uM |
| Calu-1 | 4 | uM |
| HL-60 | 5 | uM |
| MCF-7 | 8 | uM |
| Hep G2 | 20 | uM |
| CCD 39 Lu | >100 | uM |
| CCD 18 Co | >100 | uM |
| WI 38 | >100 | uM |

Various concentrations of Zn-charged fetuin prepared as described were incubated with cell lines for 6 hr. Percent of cell under apoptosis was determined by Hoechst staining and confirmed by MTS assay. The concentration of Zn-charged fetuin for the induction of 50% of cells under apoptosis (LD50) was determined. Each data represents the average of three assays.

(IV) Fetal, But Not Mature Fetuin Contains Apoptosis-Inducing Activity.

Fetuin is a fetal protein in the sense that the highest concentration was found in embryo. Fetuin is expressed at very high levels throughout the long gestational period of bovine and accounts for up to 45% of the total fetal serum proteins. However, the concentration of Fetuin in sheep and bovine dramatically decreases, probably within a few days after birth, to 1-2% of the fetal levels. Although the quantitative change of fetuin during development seems to be obvious, the qualitative change of Fetuin during the development is still unclear. To test whether the apoptosis-inducing activity of Fetuin changed during development, we isolated fetal fetuin and mature fetuin from fetal bovine serum and mature bovine serum respectively. The purified fetal fetuin and mature fetuin show similar but not the identical elution profiles on hydroxyapatite chromatography and on anionic exchange chromatography. However, they have significantly different elution profiles on ConA Chromatography, suggesting they may have different carbohydrate profiles in fetal and mature Fetuin. Interestingly, we found that while the fetuin isolated from fetal bovine serum strongly induced apoptosis in LNCaP cells, the fetuin isolated from mature bovine serum at the same concentration showed no activity inducing apoptosis. We also found that human fetuin (Alpha 2-HS glycoprotein) isolated from adult human serum fails to induce apoptosis in LNCaP cells, suggesting that mature fetuins are commonly ineffective in inducing apoptosis.

B. Test of Fetal Fetuin in a P388 Leukemia Model

All mice used were kept according to NIH standard regulation. The DBA/2 mice were divided into 4 groups, 10 mice in each group. The P388 innoculum tumor was induced by intraperitoneal injection of 106 cells per 0.1 ml innoculum. All doses were made up to a volume of 0.5 ml. Treatment was intraperitoneal injection of 0.5 ml saline (control), 0.002 ml of 10 mg/ml Fetuin (Group I), 0.02 ml (Group II) and 0.2 ml (Group III) for 10 days following tumor transplant. Mortalities were recorded daily. Results are expressed as the percent increase in life span (ILS) in Table 2.

ILS=100×Median Life Span Treated−Median Life Span Control

### Life Span Control

TABLE 2
Test of Fetuin in P388 Leukemia Model

| | | | | | (ILS) Increased |
|---|---|---|---|---|---|
| Group | No. Mice | Dose | | Survivors | Life Span |
| I | 10 | 0.5 | ml saline | 0 (24) | — |
| II | 10 | 0.002 | ml | 1 (31) | 29% |
| III | 10 | 0.02 | ml | 1 (29) | 17.2% |
| IV | 10 | 0.2 | ml | 8 (58) | 141 |

Treatment of Fetuin in highest doses have 8 mice out of 10 surviving at day 58, compared to no mice surviving in the control group. The highest dose group will increase the P388 leukemia-bearing mice life span by 141%.

C. Test of Fetal Fetuin in a Prostate Tumor Inhibition Model

The 30-35 g nude adult male mice were used for this study. The inoculum of PC-3 cells applied to induce tumor formation was adjusted with RPMI-1640 to 2×107 cells/0.1 ml. To induce tumor formation, a 0.1 ml suspension of PC-3 was injected subcutaneously at a site between the ears using a 26 gauge, ⅜″ needle. The tumors were allowed to grow for 38 days. The mice in the control group received 0.1 ml saline intraperitoneally for 10 days. Mice in the treated group received 10 consecutive 0.1 ml intraperitoneal injections of Fetuin adjusted by dilution in sterile deionized water to deliver dose concentration of 0.1 ml, 0.01 ml and 0.001 ml at 10 mg/ml. Tumors were weighed individually when the mice were sacrificed for subsequent histopathology study. Results are shown in Table 3.

TABLE 3
Test of Fetuin in Prostate Tumor Inhibition Model

| | | | | Avg. Weight |
|---|---|---|---|---|
| | | Total Weight | | Tumor (g) |
| Concentration × | Avg. Weight | Tumor (g) | | (Total # mice |
| regime | mice (g) | (Total # mice) | | w/Tumors) |
| Control | 30.33 | 9.78 | (10) | 1.63 |
| 0.2 ml × 10 | | | | |
| Fetuin | 33.9 | 6.1 | (3) | 2.03 |
| 0.001 ml × 10 | | | | |
| Fetuin | 35.3 | 6.24 | (3) | 2.08 |
| 0.01 ml × 10 | | | | |
| Fetuin | 32.0 | 3.04 | (3) | 1.01 |
| 0.1 ml × 10 | | | | |

Treatment of fetuin in the highest doses will reduce the prostate tumor size to 40% compared to the control group.

Another set of experiments were designed using immunodeficient mice (TACN: NH (S)—NuFDF homozygous males 3-4 wks old). The 2×107 PC-3 cells were implanted and allowed to grow for 6 wks. The mice in the control group received 0.5 ml saline intraperitoneally for 5 days and in the treated group received 0.5 ml Fetuin for 5 days. Results are shown in Table 4.

TABLE 4
Fetuin Inhibit Prostate Tumor in Immunodeficient Mice

| | Ave. Mouse | Ave. Tumor | Tumor Incidence |
|---|---|---|---|
| Treatment | Weight (gm) | Weight (gm) | (%) |
| Control (13 mice) | 29.7 | 2.34 | 100 |
| 0.5 ml saline for 5 days | | | |
| Treated (9 mice) | 30.6 | 0 | 0 |
| 0.5 ml Fetuin for 5 days | | | |

Fetuin was proved to reduce the Prostate tumor up to 100% in immunodeficient mice at 5 mg/30 g body weight by 5 days consecutive ip injection.

Determination of LD₅₀ of Fetuin in Rats

The Sprague-Dawley albino rats weighed 170-210 g and were divided into 4 groups, each group including 5 males and 5 females. The control group received 1 ml of saline intraperitoneally for 5 days. The other three groups were given 8.35 mg, 25 mg and 250 mg per kg body wt. of Fetuin 0.1 g/ml. All volumes were made up to 1 ml with sterile pyrogene free saline. The observation period was a least 14 days. If a visible toxic reaction occurs the observation period may be increased until visible signs of toxicity disappear. If any deaths occur during this period, the animal will be immediately autopsied to determine cause of death. During the observation period a careful clinical examination is made each day. These observations include skin and fur, eyes and mucous membranes, respiratory system, circulatory system, autonomic and central nervous system, somatomator activity and behavior patterns. Particular attention will be directed to observation of tremors, convulsions, salivation, diarrhea, lethargy, sleep or coma. Individual weights of the animals are determined shortly before the test substance is administered, and weekly thereafter. Any weight changes are recorded. At the end of the test, the surviving animals are weighed and sacrificed. Prior to sacrifice, blood is drawn from each animal to determine the serum levels of fetuin. After sacrifice, a gross necropsy is done on all animals. Any gross pathological changes are recorded and pertinent tissues preserved in 10% formalin for subsequent histopathology. The observations recorded are tabulated to see if there is any correlation between mortality and/or symptoms and the dose. The LD₅₀ for each sex is determined at 14 days, using the method of Litchfield and Wilcoxon. A dose mortality curve and slope is determined if data justify it. A table of body weight gains or loses is constructed. Necropsy finding is reported.

The results are shown in the following Table 6 and 7. There was no mortality associated with the doses administered. There appeared to be no differences in the response of the sexes. The weight gains between the saline control groups and the 250 mg/kg Fetuin in the high dose are comparable as shown in Table 6. Table 7 is a comparison of the spleen and thymus weights of the various test groups compared to the controls. The average weights of the spleen and thymus are similar. There are no significant differences. Necropsy on all of the rats appeared to be normal. No abnormal findings were seen.

TABLE 6
Average Weight Gain of Rats in Fetuin LD50 Study

| | | | | | Wt. Gain |
|---|---|---|---|---|---|
| | | | Initial Wt. | Final Wt. | Total |
| Group | Treatment | | (gm) | (gm) | (gm) |
| I. 5 male | 1.0 | ml Saline | 227.8 | 275.8 | 48.0 |
| I. 5 female | 1.0 | ml Saline | 184.0 | 204.5 | 20.5 |
| II. 5 male | 8.35 | mg/kg Fetuin | 233.5 | 283.7 | 50.2 |
| II. 5 female | 8.35 | mg/kg Fetuin | 170.2 | 203.5 | 33.3 |
| III. 5 male | 25 | mg/kg Fetuin | 174.5 | 227.5 | 53.0 |
| III. 5 female | 25 | mg/kg Fetuin | 190.3 | 218.3 | 28.0 |
| IV. 5 male | 250 | mg/kg Fetuin | 186.5 | 253.3 | 66.8 |
| IV. 5 female | 250 | mg/kg Fetuin | 164.2 | 201.7 | 37.5 |

TABLE 7
Average Weight of Thymus and Spleens of Rats Given
Different Doses of Fetuin

| Group | Treatment | | Spleen Wt. (gm) | Thymus Wt. (gm) |
|---|---|---|---|---|
| I. 5 male | 1.0 | ml Saline | 0.673 | 0.556 |
| I. 5 female | 1.0 | ml Saline | 0.561 | 0.519 |
| II. 5 male | 8.35 | mg/kg Fetuin | 0.745 | 0.599 |
| II. 5 female | 8.35 | mg/kg Fetuin | 0.513 | 0.481 |
| III. 5 male | 25 | mg/kg Fetuin | 0.796 | 0.525 |
| III. 5 female | 25 | mg/kg Fetuin | 0.714 | 0.485 |
| IV. 5 male | 250 | mg/kg Fetuin | 0.777 | 0.595 |
| IV. 5 female | 250 | mg/kg Fetuin | 0.578 | 0.619 |

Hematological and Pathological Assessment of Fetuin in Balb/C Mice

The Balb/C mice weighing 20-30 g at six to eight weeks of age were divided in six animals per group, 3 female and 3 male, in this study. Fetuin is administered IV or IP at dose of 5 mg, 0.5 mg, 0.05 mg and 0.005 mg per 20 gm mouse for 7 days. Because the nutritional supplement has potential beneficial effects on the immune response, potential toxicity is determined by clinical hematological and classical pathological parameters.

Phase I

Pre-study assessment of highest tolerated dose administration by IV or IP injection. The end-point is the highest non-lethal dose administered by each route. Animals are screened daily for signs of lethality and the total time of observation is seven days.

Phase II

Clinical and histological assessment of highest tolerated and lessor doses of Fetuin. Seven days prior to the beginning of phase II each animal is bled to determine its background values for hematological and clinical chemistry parameters. The highest tolerated dose and the next lowest, as well as the vehicle are injected in a single injection by the best-tolerated route. After 24 hours the animals are sacrificed. Gross and microscopic pathologic examination are performed on the organs. Hematological, clinical chemistries and clinical pathology tests are performed: (1) Blood Collection and Analysis: Blood samples are collected from each mouse, one at the start of the experiment prior to receiving their injection of Fetuin and the second collected at the time of euthanasia. The samples are collected via retro-orbital sinus bleed, or tail bleed, under anesthesia. (2) Hematology: Approximately 125 ul of whole blood is collected in an EDTA tube at the specified time. The following hematological parameters are determined: White blood cell count (WBC), Red blood cell count (RBC), Hemoglobin (HGB), Hematocrit (HCT), Mean Corpuscular Volume (MCV), Mean Corpuscular Hemoglobin (MCH), Mean Corpuscular Hemoglobin content (MCHC), Platelet count (PLT), WBC differential count. Whole blood samples for hematology are disposed of one week after analysis. (3) Clinical Chemistry: Approximately 125 ul of whole blood is collected in a tube containing no additives for serum at the specified times. Tests determine the following clinical chemistry parameters: Alanine aminotransferase (ALT) and Aspartate aminotransferase (AST). (4) Collection of Tissue: The following tissues are collected into 10% neutral buffered formalin (NBF): kidney, liver, lung, thymus, reproductive organ, heart, spleen and lymph nodes. Any abnormalities or gross pathology found at necroscopy are recorded for each animal. (5) Histopathology: Histology is done on the tissue samples submitted for each animal. The tissues will be paraffin-embedded, sectioned and stained with hematoxylin and eosin (H&E). A pathology reads the slides for apparent abnormalities.

Phase III

Clinical and histological assessment of highest tolerated dose of Fetuin administered daily over 14 days. The highest tolerated dose as determined in Phase II and the vehicle is injected daily for fourteen consecutive days by the best-tolerated route. After 24 hours, one-half of the animals are sacrificed. The remainder are sacrificed after 14 days. Gross microscopic pathologic examination are performed on the organs recovered from all animals. (1) Blood collection and analysis: Blood sample is collected from each mouse, one at the start of the experiment prior to receiving their injection of Fetuin and the second collected at the time of euthanasia. The samples are collected via retro-orbital sinus bleed or tail bleed under anesthesia. (2) Hematology: Approximately 125 ul of whole blood is collected in an EDTA tube at the specified time. The following hematology parameters is determined: White blood cell count (WBC), Red blood cell count (RBC), Hemoglobin (HGB), Hematocrit (HCT), Mean Corpuscular Volume (MCV), Mean Corpuscular Hemoglobin (MCH), Mean Corpuscular Hemoglobin Content (MCHC), Platelet count (PLC), WBC differential count. Whole blood samples for hematology are disposed of one week after analysis.

(3) Clinical Chemistry: Approximately 125 ul of whole blood is collected in a tube containing no additives for serum at the specified times. The following clinical chemistry parameters are assessed: Analine aminotransferase (ALT), Aspartate aminotransferase (AST), Blood urinary nitrogen content, Cholesterol level and lipemic index.(4) Collection of tissue: The following tissues are collected into 10% neutral buffered formalin (NBF): kidney, liver, lung, thymus, reproductive tissue, heart, spleen and lymph nodes. Any abnormalities or gross pathology found at necroscopy are recorded for each animal. (5) Histopathology: Histology is done on the tissue samples submitted for each animal. The tissue is paraffin-embedded, sectioned and stained with hematoxylin and eosin (H&E). A pathologist reads the slides for any apparent abnormalities.

The observation and analysis results were shown as the following. Mice injected with 5 mg, 0.5 mg, 0.05 mg and 0.005 mg of Fetuin via IP route survived for 7 days without any signs of distress, lethargy, ruffled coat, decreased activity, abnormal behavior, dyspnea, unconsciousness, or seizures. No lethality and other gross macroscopic lesions were observed in any organs such as livers, kidneys, spleens, lungs and small intestines in any of the treatment groups. Ten animals from each dosage group had their tissues examined for microscopic structural changes. All tissues from all animals were normal in architecture. No evidence of fibrosis and sclerosis were observed in all tissues. Only rarely did the liver and lung show small foci of inflammatory cells without pattern related to dose or location. There is no significant body weight change in the control group and Fetuin treated group. No significant differences in White Blood Cell (WBC) levels 24 hrs after injection of various doses were observed among the treatment and control groups [p=0.126]. All WBC values were within the normal limits. Blood glucose levels were significantly elevated [P=0.03] in the animals given 5 mg Fetuin (41.6 mg/dL) compared to the 0.005 mg per animal dose (13 mg/dL). The glucose levels of the animals receiving intermediate doses of Fetuin 0.5 mg and 0.05 mg were 25 and 27 mg/dL, respectively. The levels of Blood Urinary Nitrogen (BUN) were elevated in the highest and lowest dose groups. The pattern of differences between treatment groups appeared unrelated to dose. The AST (Aspartate Aminotransferase) and ALT (Alanine Aminotransferase) liver enzyme levels were within the normal limits for Balb/C mice. [P=0.306 and P=0.444, respectively]. The hematocrit values for each group ranged from 41.2 to 42% which is well within the normal range for Balb/c mice of this sex and age. No differences were observed between groups [P=0.569]. Animals treated with 5 mg per mouse had cholesterol levels elevated by 14% compared to the lower doses. The highest dose were significantly different from all the lower doses. [P<0.05]

The overall conclusion from this study is that Fetuin has very low toxicity in the Balb/c mouse as judged by modest changes in some chemistry values and the fact that microscopic anatomy of the major organ systems was completely within normal parameters. Although the pharmacokinetics study has not elucidated, the histopathological data showed that there was no obvious architectural damage in various tissues. If Fetuin is administered at doses higher than 250 mg/kg body weight, these data suggest that follow-up testing for cholesterol and blood glucose may be warranted. This study suggests that there is no evidence of acute or chronic toxicity of Fetuin even when administered acutely or chronically.

Rabbit Study to Determine the Safety of Fetuin by Intravenous Administration

Rabbits weighing 1.75 to 2.5 kg are used in the study, 2 rabbits/group. The control group receives 1 ml of saline. The treated group is administered intravenously by 0.01 ml, 0.1 ml and 1 ml per kg body weight of Fetuin 0.175 g/ml. All dilutions are made with sterile pyrogen free saline (0.9%). The study is designed to limit to an acceptable level the risk of a febrile reaction in patients. The animals are given their respective doses of Fetuin at two-day interval for a total of three doses if no reaction occurs. Blood samples are collected for a baseline prior to treatment and then 24-36 hours after the third treatment. At the end of the treatment period, blood is drawn to perform hematology to compare with the baseline levels. The body temperature is recorded for three hours post injection to determine if a febrile reaction is produced. The body temperature is monitored prior to injection (30 min) and then at 30 min interval for three hours. Prior to sacrifice, blood is drawn from each animal to determine the serum level of Fetuin. Organs are examined in all animals. The target organs: spleen, lymph nodes, thymus are examined for evidence of increased size. All tissues excised are preserved in 10% formalin for subsequent histopathology if necessary. Observations made at each dose level are tabulated to see if there is any correlation of symptoms and/or mortality and dose level of the test material. Body weights and necropsy results are recorded. Rabbit body temperature increases for each dose level is reported and conclusions made from the study.

The observation of rectal temperature change of rabbit over 3 hr post injection period indicated that any dosages of Fetuin administration did not cause body temperature increase. (Table 8) None of the animals displayed any signs of distress during the test.

TABLE 8
Effects of Fetuin on the Body Temperature Increase in
Rabbits in Which the Temperature Was Monitored 3 hours Post
Intravenous Injection.

| | | Mar. 6, 2000 | Apr. 6, 2000 |
|---|---|---|---|
| Dose of Fetuin, mg/kg | Rabbit # | Temp Increase | Temp Increase |
| Control | 1 | 0 | 0 |
| | 2 | 0 | 0 |
| 65.7 mg/kg | 3 | 0 | 0 |
| | 7 | 0 | 0 |
| 126 mg/kg | 5 | 0 | 0 |
| | 6 | 0 | 0 |
| 185 mg/kg | 8 | 0 | 0 |
| | 4 | 0 | 0 |

Table 9 summarizes the body weight gains of the rabbits over a 7 week period. There are no apparent differences that are related to the dose of Fetuin administration.

TABLE 9
Body Weight Gain of Rabbits Given Different Doses of Fetuin

| Dose of | | Wt. Gain | Wt. Gain | Wt. Gain | Wt. Gain | Mean of |
|---|---|---|---|---|---|---|
| Fetuin, mg/kg | Rab# | Mar. 6, 2000 | April 6, 2000 | April 19, 2000 | (kg) | Wt.Gain |
| Control | 1 | 2.65 | 3.10 | 3.71 | 1.06 | 1.17 |
| | 2 | 2.73 | 3.20 | 4.02 | 1.29 | |
| 65.7 mg/kg | 3 | 2.48 | 3.15 | 3.65 | 1.17 | 1.14 |
| | 7 | 2.61 | 3.28 | 3.72 | 1.11 | |
| 126 mg/kg | 5 | 2.60 | 3.31 | 3.56 | 0.96 | 0.76 |
| | 6 | 2.70 | 3.10 | 3.26 | 0.56 | |
| 185 mg/kg | 8 | 2.80 | 3.19 | 3.69 | 0.89 | 1.02 |
| | 4 | 2.61 | 3.26 | 3.77 | 1.16 | |

The variation in blood glucose levels is shown in Table 10. The control rabbit had a non-fasting blood glucose level average of 207 (range 257-158), whereas at 65.7 mg Fetuin/kg body weight the average glucose level was 91 mg/dl. At 126 mg/kg body weight of Fetuin, the average blood glucose was 172 mg/dl and at 185 mg/kg of Fetuin the average blood glucose was 103 mg/dl.

TABLE 10
Blood Glucose mg/dl Non-fasting Values

| Dose of Fetuin | | Blood Glucose Level | Mean of blood |
|---|---|---|---|
| mg/kg | Rabbit # | mg/dl | Glucose level |
| Control | 1 | 257 | 207 |
| | 2 | 158 | |
| 65.7 mg/kg | 3 | 94 | 91 |
| | 7 | 88 | |
| 126 mg/kg | 5 | 221 | 172 |
| | 6 | 123 | |
| 185 mg/kg | 8 | 117 | 103 |
| | 4 | 89 | |

The total serum protein, albumin, globulin and albumin/globulin ratios is summarized in Table 11. The total serum protein levels are all within normal range. The total albumin is elevated in all groups including the control group. With the low globulin levels, this gives an abnormal albumin/globulin ratio. This was also seen in the saline controls. We have no explanation for this unusual observation, but it has no relationship to the dose of Fetuin.

TABLE 11
Total Serum Proteins, Albumin, Globulin and Albumin/Globulin Ratio

| Dose of | | | | | Albumin/ |
|---|---|---|---|---|---|
| Fetuin, | | Ttl Serum | | | Globulin |
| mg/kg | Rabbit # | Protn, g/dl | Albumin | Globulin | Ratio |
| Control | 1 | 5.7 | 5.2 | 0.5 | 10.4 |
| | 2 | 6.0 | 5.7 | 0.3 | 19.0 |
| 65.7 mg/kg | 3 | 6.1 | 5.6 | 0.5 | 11.2 |
| | 7 | 5.9 | 5.5 | 0.4 | 13.8 |
| 126 mg/kg | 5 | 5.9 | 5.3 | 0.6 | 8.8 |
| | 6 | 5.9 | 5.3 | 0.6 | 8.8 |
| 185 mg/kg | 8 | 6.1 | 5.2 | 0.9 | 5.8 |
| | 4 | 5.6 | 5.1 | 0.5 | 10.2 |

*Note: Even the control rabbits had elevated A/G ratios.

The Blood Urea Nitrogen (BUN) level of the rabbits is shown in Table 12. The control mean BUN was 20.5 mg/dl, whereas the high dose fetuin (185 mg/kg body weight) had a mean value of 20.5 mg/dl.

TABLE 12
Blood Urea Nitrogen of Various Fetuin Treatment

| Dose of Fetuin, | | Blood Urea Nitrogen, | |
|---|---|---|---|
| mg/kg | Rabbit # | mg/dl | Mean of BUN |
| Control | 1 | 20 | 20.5 |
| | 2 | 21 | |
| 65.7 mg/kg | 3 | 21 | 22.5 |
| | 7 | 24 | |
| 126 mg/kg | 5 | 20 | 18.5 |
| | 6 | 17 | |
| 185 mg/kg | 8 | 19 | 20.5 |
| | 4 | 22 | |

The circulating liver enzymes ALT (Alanine transferase) and AST (Aspartate transferase) in the rabbits treated with Fetuin was summarized in Table 13. Although some of the individual rabbit values are elevated there appears to be no dose relationship.

TABLE 13
ALT and AST Liver Enzyme Levels in Rabbits
Treated Intravenously With Different Doses of Fetuin.

| Dose of Fetuin | Rabbit | Alanine | Mean of | Aspartate | Mean of |
|---|---|---|---|---|---|
| mg/kg | # | Transferase | ALT | Transferase | AST |
| Control | 1 | 10 | 30.5 | 58 | 45.5 |
| | 2 | 51 | | 33 | |
| 65.7 mg/kg | 3 | 29 | 41.5 | 32 | 30 |
| | 7 | 54 | | 28 | |
| 126 mg/kg | 5 | 42 | 37 | 37 | 51.5 |
| | 6 | 32 | | 66 | |
| 185 mg/kg | 8 | 49 | 42.5 | 26 | 24.5 |
| | 4 | 36 | | 23 | |

The cholesterol values shown in Table 14 vary from a low of 32 mg/dl to a high of 71 mg/dl. There is no apparent correlation between the cholesterol values and the dose of Fetuin.

TABLE 14
Cholesterol Values for Rabbits Treated With Different Doses of Fetuin

| Dose of Fetuin, mg/kg | Rabbit # | Cholesterol, mg/dl | Mean |
|---|---|---|---|
| Control | 1 | 32 | 43 |
| | 2 | 54 | |
| 65.7 mg/kg | 3 | 69 | 70 |
| | 7 | 71 | |
| 126 mg/kg | 5 | 49 | 57.5 |
| | 6 | 66 | |
| 185 mg/kg | 8 | 35 | 45.5 |
| | 4 | 56 | |

The white blood cell count varied from 9.2 to 6.7×103/ml. There appears to be no increase due to the dose of Fetuin. (Table 15)

TABLE 15
White Blood Cell Counts in Rabbits Treated with Various Doses Fetuin

| Dose of Fetuin, mg/kg | Rabbit # | WBC, K/ul | Mean of WBC |
|---|---|---|---|
| Control | 1 | 9.2 | |
| | 2 | 7.9 | 8.55 |
| 65.7 mg/kg | 3 | 6.9 | |
| | 7 | 10.1 | 8.5 |
| 126 mg/kg | 5 | 9.5 | |
| | 6 | 6.7 | 8.1 |
| 185 mg/kg | 8 | 10.7 | |
| | 4 | 6.8 | 8.75 |

Table 16 shows that the hemoglobin and hematocrit are similar in the control as well as the treated groups. There is no relationship to the dose of Fetuin even in the groups having a highest dose of Fetuin.

TABLE 16
Hematocrit and Hemoglobin of Rabbits Treated with Fetuin

| Dose of Fetuin | | | | Hemoglobin | |
|---|---|---|---|---|---|
| mg/kg | Rabbit # | Hematocrit | % Mean | g/dl | Mean |
| Control | 1 | 39.5 | 37.4 | 12.8 | 12.1 |
| | 2 | 35.3 | | 11.5 | |
| 65.7 mg/kg | 3 | 37.0 | 38.6 | 12.6 | 13.0 |
| | 7 | 40.2 | | 13.4 | |
| 126 mg/kg | 5 | 38.2 | 38.4 | 12.4 | 12.6 |
| | 6 | 38.6 | | 12.9 | |
| 185 mg/kg | 8 | 35.2 | 36.3 | 11.4 | 11.8 |
| | 4 | 37.3 | | 12.3 | |

The spleen and thymus weight in each group is recorded as in Table 17. There is no correlation between the dose and the small variations in total weights of these organs.

TABLE 17
Spleen and Thymus Weights
of the Rabbits Given Two Doses of Fetuin

| | | Spleen | | | |
|---|---|---|---|---|---|
| Dose of Fetuin | Rabbit # | Wt. (g) | Mean | Thymus Wt. (g) | Mean |
| Control | 1 | 1.32 | 1.39 | 4.69 | 4.89 |
| | 2 | 1.46 | | 5.10 | |
| 65.7 mg/kg | 3 | 1.54 | 1.63 | 6.56 | 5.85 |
| | 7 | 1.72 | | 5.21 | |
| 126 mg/kg | 5 | 1.37 | 1.43 | 4.90 | 5.05 |
| | 6 | 1.9 | | 5.21 | |
| 185 mg/kg | 8 | 1.71 | 1.67 | 5.26 | 5.43 |
| | 4 | 1.64 | | 5.60 | |

In conclusion, Fetuin has no toxicity in these studies. The changes observed in some of the serum chemistries were minor and did not correlate with the dose of Fetuin. The observation of the lack of a pyrogen response on successive injections of Fetuin indicates that it can be given repeatedly to the animals. The spleen and thymus weights did not show any dose related inhibition or acceleration in growth.

To Determine the Effects of Different Doses of Fetuin on the PC-3 (Human Prostate Tumor) Cell Line in Immunodeficient Nude Mice

The mice divided into 5 each group in this study were TACN:NH (S)—NuFDF homozygous males 3-4 weeks old and weighed an average of 22 g at the start of the experiment and 30 g at the termination of the experiment six weeks later. The animals were induced to prostate tumor formation by inoculating 2×107 PC-3 cells in 0.1 ml PBS suspension on the upper half of the dorsal thorax of the mouse. The tumor will be allowed to grow for 6 weeks. The mice in the control group will receive 0.1 ml saline intraperitoneally for 5 days and the mice in the treated group will receive 0.02 ml, 0.1 ml and 0.2 ml of Fetuin 10 mg/ml per day for 5 days. Treatment will start the day after tumor inoculation. Tumor will be weighed at the end of the experiment when the mice will be sacrificed for subsequent histopathology. Animals will be monitored daily for signs of tumor and/or any toxic effects from the test material. [The result was shown in Table 4]

Potential Immunosuppression of “Fetuin Protein” in Balb/C Mice

The objective of this study is to determine whether or not administration of Fetuin induces changes in the immune system which leads to alterations of the secondary lymphoid organs and populations of immune cells, as well as functional changes in the immune system. The immune system, with ongoing cell proliferation and differentiation, as well as gene amplification, make it highly susceptible to toxic insults. The cell populations which are the most susceptible to the overt action of immunotoxic agents are the thymus and bone marrow populations (site of lymphoiesis). The decrease or disappearance of any of these cell populations is often the indicator of immunotoxicity. In general, the secondary lymphoid organs are usually less susceptible to toxic insult compared to the cells that inhibit the thymus and bone marrow. Toxicity to the tissue framework progresses through the sequential steps of degeneration and atrophy ending in fibrosis. It is important to note that potential agents often produce immunotoxicity at concentrations less than those causing weight loss and/or histopathological tissue alteration. Time of specimen sampling relative to administration of the drug is extremely important when dealing with immune cells. The immune system with its high regenerative capacity, can restore cells populations in a relatively short time. Therefore, the validity of immunosuppression analysis is highly dependent on the time interval between drug treatment and analysis.

A two tier approach to assess immunotoxicity or immunosuppression has been developed. Tier I tests include, but are not limited to:

1. Blood counts (see Hematological and Pathological Assessment)

2. Differential blood counts (see Hematological and Pathological Assessment)

3. Examination of spleen, liver, and kidney for pathological alterations (see Hematological and Pathological Assessment)

4. Examination of spleen and lymph nodes for immune depletion

5. Primary immune response to sheep red blood cells (SRBC)

Tier II tests include, but are not limited to:

1. Quantitation of T and B cells by flow cytometric analysis

2. Secondary immune response to SRBC

3. Host resistance challenge models (syngeneric tumor challenge or parasite and bacterial infectivity challenge)

Material:

The Balb/C mice weighed 20-30 g with six to eight weeks of age and divided equally between male and female are used. Fetuin is administered IV or IP to each group of six animals either a single dose or multiple doses ranging from 0.05 mg to 5 mg per 20 g body weight. The volume of each injection is 0.2 ml. Phosphate buffered saline is used as the control and dilution vehicle.

Primary and secondary immune responses to SRBC: Sheep red blood cells are purchased from Sigma Chemical Co. Guinea pig serum are used as a complement source. The Guinea pig serum absorbs 3× against 20% (V/V) of washed SRBC at 40 C for 30 min to remove any endogenous or cross-reacting antibodies to SRBC. The absorbed guinea pig serum is aliquoted into 1 ml volumes and frozen at −80° C. for use in the immunosuppression assay. Agarose suitable for the plaque forming (PFC) assay will be used.

Flow cytometric characterization of splenic T- and B-cells in Fetuin treated mice: T cells of mice can be recognized by the presence of Thy 1.2 or Thy 1.1 (depending on the genotype of the mouse), receptors isolated on the surface of peripheral T cells (lymph node, spleen and blood) as well as thymic epithelia. Peripheral B-cells are recognized by the presence of surface IgM (sIgM and/or IgD (sIgD) or IgG (sIgG). Though other markers exist to identify B-cells, sIg detection is inexpensive and relatively easy to perform. Fluoresceinated anti-Thy 1.2 and rhodamine goat anti-mouse IgM (sIgM) and/or goat anti-mouse light chains (k+) (sIg) are purchased. Fitc (fluoresceinated) anti-Thy 1.1 (opposite genotype Balb/C strain) will be used to determine background levels of fluorescence on T-cells. Fluoresceinated of rhodaminated antibodies to antigen not found on the cell surface, such as albumin, will be used to determine the background level of fluorescence on B cells.

Protocol:

Validation of Immune Response to Sheep Red Cells:

Primary Response:

Control mice are injected either IP or IV with 107 washed SRBC on day 0. After 5 days, 4 mice from this group are sacrificed and their spleens removed. Single cell suspensions prepared from the spleen are assayed against SRBC lawns with and without properly diluted guinea pig complement at 370 C for 1 hr. The number of antibody forming cells or plaque forming cells (PFCs) per million cell plated are calculated as well as the total number of PFCs per spleen. The predominant, if not exclusive, type or class of antibody detected by this technique is IgM.

Secondary Response:

The secondary immune response to SRBCs is highly T cell (Th2) dependent. Mice which have been previously immunized as outlined above are allowed to rest for 28 days. On day 29, each animal in the primary response measurement receives 107 washed SRBCs by either IP or IV injections. Starting on day 30 through 40, 4 mice each are sacrificed and the spleen assayed for the number of IgM and IgG PFCs per million cells and total number of IgM and IgG PFCs per spleen. To detect the IgG response to SRBC, an additional reagent, rabbit anti-mouse IgG (Fc specific antiserum) is added. This reagent detects mouse IgG antibodies, secreted by an antibody secreting cells, bound to SRBCs. In addition, the reagent initiates the complement dependent lysis of the SRBC. A lysis of SRBCs about the secreting lymphocyte or plasma cell produces a clear zone (plaque) in the red cell lawn. The system determines the magnitude of the responses as well as the temporal appearance of the antibody responses to SRBCs.

Effects of Fetuin on Primary Immune Responses to SRBC:

Fetuin will be injected IP at dosage of 5 mg to 0.005 mg per 20 g mouse at days −3, −2, −1 and 30 min prior to SRBC primary immunization and day +1. An equal number of animals will be injected on the same time schedule with vehicle. Starting at day +4 through +8, 4 mice at each time point will be sacrificed and the IgM PFC response determined as outlined above. There are two potential responses: Firstly, the Fetuin will have no adverse affect on the IgM PFC response. It will have the same magnitude and temporal appearance as control or vehicle injected mice. Secondly, the Fetuin will delay or diminish the appearance to IgM PFC response, If the response is delayed but eventually reaches the same magnitude as the control response, this would suggest that Fetuin delays certain signals within SRBC specific B cells and/or T cells or accessory cells. If greatly diminished, Fetuin may inhibit the delivery of signals between and/or within specific B- and T-cells. If a significant level of inhibition is observed in the primary response to SRBC by Fetuin administration, it would be expected that the secondary response would be also significantly reduced. Cellular events taking place during the primary response set the stage for subsequent recruitment and activation of memory cells in the secondary response.

Effects of Fetuin on the Secondary Immune Response to SRBC:

To investigate the effects of Fetuin administration on the secondary immune response to SRBC, an adequate number of mice will be immunized as outlined above (Primary immune response). Starting on day +27, +28 and 30 min prior to secondary challenge with SRBC (day +29) and on day +31, mice will be injected with a dosage of 5 mg to 0.05 mg Fetuin per 20 g mouse or control vehicle. Starting on day +32 through day +38, 4 mice each will be sacrificed and the IgM and IgG PFCs to SRBC determined. ANOVA and other appropriate statistical tests will be performed on the data to examine whether statistically significant differences between the experimental and control groups exist. If Fetuin does not affect the secondary immune response, then the numbers of IgG synthesized antibodies specific to SRBC will be equivalent to that of the control animals (Uninjected or vehicle injected animals). The temporal appearance of antibody forming cells will also be the same as in the control animals. If Fetuin delays communication between cells, a delay in the peak of the antibody forming cells (PFC) may be observed. On the other hand, Fetuin could significantly inhibit the class shift from IgM to IgG antibody. This class change is T-helper cell dependent, since the class shift of IgM to IgG depends on secretion of IL-4 and IL-5. Thus, T-helper cell function could be normal (normal levels of IL-4 and IL-5), but the B-cells may be unresponsive to T-cell signals. In this case, more complicated experiments using adoptive transfer of responsive B-cells or T-cells to an irradiated host may be necessary in order to define the exact cellular mechanisms.

Quantitation of T- and B-Cells by Immunophenotyping

Two separate approaches to determine the effects of Fetuin on T-cells and B-cells in the spleen will be undertaken: an acute or short-term response to Fetuin and a chronic or long-term exposure to Fetuin.

For the short-term exposure, groups of 4 mice will be injected IP with 5 mg to 0.05 mg Fetuin per 20 g mouse or control vehicle for 3 days. Starting on day +4 through +10, 4 animals will be sacrificed. Spleen will be removed and the proportions and absolute numbers of T-cells (Thy 1.2 bearing cells) and B-cells (sIgM+sIgG bearing cells) will be calculated. Furthermore, the log 10 geometric mean levels of Thy 1.2 and sIg expression will be determined. Changes (increase) in the levels of surface expression of Thy 1.2 or sIg may be indicative of the appearance of immature T-cells and B-cells in the spleen. These immature cells may not function properly in the peripheral or secondary lymphoid tissues.

For the long-term exposure, groups of 4 mice will be injected IP with 5 mg Fetuin per 20 g mouse or control vehicle for 14 days. Starting on day +15 through +30, 4 mice will be sacrificed every other day and the analysis performed as outlined in the short-term protocol. Long-term exposure to Fetuin may reveal a direct toxic effect on the primary lymphoid tissues of thymus and bone marrow where T-cells and B-cells are produced, respectively. A slow depletion of cells from the spleen would indicate inhibition of primary lymphoiesis (thymus and bone marrow). Therefore, lymphocyte populations in the spleen are not being replaced and the spleen becomes depleted by normal loss of lymphocytes. On the other hand, if the depletion is rapid and dramatic, a possibility exists that Fetuin is directly toxic to splenic T- and B-cells. If this were the case, cessation of the drug should permit complete restoration of the cell populations over a period of several weeks.

Outline of Surface Staining of Splenocytes

1. Add 100 ul of single suspension of splenocytes (1×106) from each animal to a series of separate 12×75 mm tubes. Cells are suspended in Balanced Salt Solution (BSS) containing 0.1% azide and 1% bovine serum albumin (BSA).

2. Add 20 ul-50 ul of surface specific labeled antibodies to one set of tubes. To another set of tubes, add 20 ul-0.50 ul appropriate control antibodies for background surface staining.

3. Mix thoroughly and incubate for 15 to 30 min at room temperature (20-250 C).

4. Add 100 ul of Cal-lyse (maintained at room temperature) and incubate for 5 min.

5. Incubate for 10 min at room temperature.

6. Add 1 ml of deionized distilled water and incubate for 5 min.

7. Analyze cells on flow cytometer or store samples in the refrigerator until analysis is performed.

8. The log 10 geometric mean of surface fluorescence for each specific antibody will be calculated. In addition, the relative proportions of T- and B-cells, and the absolute number of T- and B-cells, will be calculated for each group of animals.

Statistical Analysis:

Statistical analysis is performed using SigmaStat version 2 and graphical representations of the data is done using SigmaPlot version 4.

Results:

Spleen-to-Body Weight Ratios:

Animals were injected with varying doses of Fetuin for 7 consecutive days and killed 24 hr later. Body and spleen weight were recorded for each animal, which permitted the calculation of spleen-to-body weight ratios. There were no significant differences in the ratios of spleen to body weight ratios between treatment groups. [P=0.301]

Density of Thy 1.2 and Surface Ig Expression and Prevalence of T- and B-Cells:

Splenocyte populations collected from animals treated with various doses of Fetuin were stained with Fitc-anti-Ig or Fitc-anti-Thy 1.2. A Coulter Elite Flow Cytometer was used to analyze the cell populations. Only lymphocyte populations were analyzed by using a gate defined by forward and right angle scatter. The fraction of cells which for s-Ig or Thy 1.2 are obtained from only the lymphocyte population. Other cells were excluded from the analyses. A multiple comparison procedure of ANOVA (Student-Newman-Keuls Method Post Hoc Test) was used for determination of Statistical differences between the treatment groups. The staining for Thy 1.2 and s-Ig revealed two separate and distinct populations of lymphocytes where region M1 contains cells bearing Thy 1.2 or s-Ig (note much higher fluorescence signal) compared to region M2. Region 2 contains cells not bearing Thy 1.2 or s-Ig where the cell populations exposed to Fitc-sheep IgG or Fitc—Thy 1.1 (controls) produced much less of a fluorescence signal.

There were differences in the fraction of lymphocytes which were B-cells (ie. s-Ig+), yet multiple comparison methods revealed no differences between treatment groups [p=0.439]. Similarly, there were no significant differences in the cell surface density of s-Ig on the B-cells from the different groups. [p=0.151] The fraction of splenic lymphocyte that were T-cells remained constant over the 104 fold differences in Fetuin doses. [p=0.365]. The mean level of expression of Thy 1.2 among the treatment groups did not differ significantly one-from another. [p=0.069]

The Effects of Two Dosages on the IgM and IgG Immune Response to Sheep Red Blood Cell Immunization:

Both the IgM and IgG responses were unaffected by pre-treatment and post-treatment with 104 range of Fetuin doses. [p.0.05] The IgG PFC was corrected for the presence of IgM PFC. (the IgM PFC represented less than 10% of the IgG PFC).

Discussion:

A multi-level approach for the detection of alterations in the immune system after treatment with a wide-range of Fetuin doses was performed. The dosing regimen was at least 7 days of consecutive IP administration of either control (saline), or 5 mg, 0.5 mg, 0.05 mg and 0.005 mg of Fetuin per mice. These doses are equivalent to 250 mg, 25 mg, 2.5 mg and 0.25 mg per kg body weight, respectively. The assays used included alterations in spleen weight to body weight reflecting changes in overall cellularity of the spleen, corrected for differences in body weight between animals. Such corrections allow accurate comparisons of cellularity of the spleen. Functional assays requiring intact antigen presenting cell populations (APC), normal activation and recruitment of naive B cells and their subsequent clonal expansion and immunoglobulin secretion was performed by the detection of IgM antibody forming cells (PFC) in the primary immune response to sheep red blood cells. A more stringent test, requiring the same parameters but with added requirements of the generation of T cell helper cells and B memory cell formation, was performed by the detection of IgG antibody forming cells (PFC) in the secondary immune response to sheep red blood cells. Depending on the timing of Fetuin administration, direct effects of Fetuin on T memory cell and B memory cell function could be examined. Finally, the use of surface immunophenotyping permitted the determination of alterations in the frequency of T- and B-lymphocytes within spleen after treatment with Fetuin.

The data revealed that after cessation of Fetuin treatment, there were no significant differences in the spleen to body weight ratios. Functional tests of IgM and IgG antibody forming cell population in response to sheep red blood cell immunization showed no deleterious effects of Fetuin doses of 250 mg/kg body weight to 0.25 mg/kg body weight, respectively.

These data suggest that the Fetuin did not negatively effect:

Antigen presenting cell populations (APC)

Recruitment of B cells

Activation of B cells

Clonal expansion of B cells

Immunoglobulin secretion of antibody forming cells

The IgG immune response to sheep red blood cells has additional requirements to the IgM immune response:

Generation of T- and B-memory cells

T-memory cells providing “help” functions to B-memory cells

Immunoglobulin IgG gene rearrangement, transcription and translation

IgG secretion

The fact that Fetuin did not decrease the specific IgG response to sheep red blood cells strongly suggests that all of the four requirements noted above were negatively impacted by Fetuin. Examination of the two major lymphoid cell populations in the spleen revealed that T and B cells were largely unaffected by Fetuin administration. The following supports this assertion: This type of analysis is time-sensitive and recovery can mask critical and significant biological differences. The design of the experiment reported here took the time factor into consideration. The animals were treated for 5 consecutive days and assayed 1 day later, when potential positive effects if have any were most likely to appear.

In conclusion, immunosuppression was not associated with any of the dosages of Fetuin used in the experiments herein reported. The dosages used in these experiments were 0.25 mg/kg body weight to 5 mg/kg body weight, respectively. These dosages offer a wide, non-toxic therapeutic window.

Pharmacokinetics and Pharmacodynamics

The mechanism of Fetuin to exhibit the drug effect is dependent on the binding of transforming growth factors and cytokines and reducing the growth signal of tumor cells at the extracellular level. Due to the nature of mechanism, no antibody and delivery problem is expected. In our assay, this binding can occur within a few minutes in the tumor cells and have no effect in normal cells overnight or even several days. Based on these reasons, pharmacokinetics concerns will not be the same as other drugs which are basically toxic. The histopathological data indicated that no architectural damage in various tissues resulting from the treatment and 14 days observation after treatment. As far as the pharmacodynamics is concerned, we suggest not to take at the same time as other drugs since the drug effect can introduce so rapidly. No metabolites or degradation is anticipated due to the nature of drug and so instantly to exert the drug effect.

E. Clinical Protocol

Decreased levels of human fetuin have been observed in patients with acute lymphocytic and nonlymphocytic leukemias, chronic granulocyte and myelomonocyte leukemias, metastasizing solid tumors, Hodgkin's and non-hodgkin's lymphomas, myelofibrosis, multiple myeloma, systemic lupus erythematosus, acute alcoholic hepatitis, chronic active hepatitis, liver cirrhosis, acute and chronic pancreatitis (5). In human hepatoma cell line HepG2, it has been shown that the cytokines IL-6 and interleukin-1beta cause the down-regulation of human fetuin synthesis and that this decrease could be observed both at the protein and mRNA level.

I. Leukemia

1. Rationale of Dosimetry

The principle of subscribing dose is not exceeding the highest tolerance safe dose in the healthy subjects. Under this rationale, we expect less side effects but achieve the drug effect. The unit to use in dosimetry is the body weight with the following explanation. The tissue culture assay is based on surface area that related to the number of cells. The body weight is more related to the dimention quantity, however, the animal model is a good prediction of physical quantity in humans. We prefer the body weight than the surface area since the estimated quantity in vivo is correlated well enough to the tissue culture assay. Of course, adjustment may be needed throughout the human clinical trial as we evaluate the routine test during the treatment. In the case of Leukemia, if we consider the volume of total blood, mice is about 1 ml whereas adult human is about 3 liter, which is 1 to 3000 ratio. If we consider the body weight, mice are 20 g whereas adult humans are about 60 kg, which is also 1 to 3000 ratio. Therefore, the rationale under body weight ratio should be very close to the estimation.

2. Symptoms and Responses

Patients with lymphoblastic leukemia present with signs and symptoms of marrow failure. These include pallor and fatigue, bleeding and bruising, and fever and infection due to anemia, thrombocytopenia and neutropenia, respectively. The risk of bleeding increases with platelet counts less than 20×105/ul and often involves skin and mucosal sites. The increased risk of infection occurs with absolute count of neutrophil counts less than 500/ul. Infections commonly involve mucosal site such as the pharynx and perianal area as well as the lung and skin (particularly intravenous line sites). Since Fetuin was found to exhibit bacteriacidal effect, the fabrile situation was predicted to be less manifestation. Patients with acute lymphoma leukemia (ALL) can present with infiltration of spleen, lymph nodes, liver, skin or CNS (particularly as a site of relapse). Patients with leukemia meningitis generally present with headache, nausea and cranial nerve pulses. Several metabolic abnormalities are seen in patients with ALL. Uric acid nephropathy is caused by the rapid turnover of acute leukemia cells. This is aggregated by dehydration, acidosis and therapy leading to tumor cell lysis. The use of hydration, alkalination of the urine and allopurinol can prevent urate nephropathy. In addition to the regular whole panel of blood analysis, a leukemia marker, CD25, is suggested to be included in analysis. The immunosuppression tier will be evaluated as in preclinical trial protocol. The exact dose will be determined by body weight, age and the analysis of transforming growth factors, cytokines and hormones representing in the designed differential display in individual patients. The number of treatments will be determined by the parameters of patient examination. The route of administration is intravenous. The trace of patient history after treatment every 1, 3 and 6 months will be used to assess the side effect of the treatment. The starting dose is suggested to be 10 mg/kg body weight and escalating as proposed in next section.

3. Safety and Toxicity Parameters

The toxicity will be monitored on a daily schedule during treatment and weekly after treatment for a month, then 3 month and 6 month. The safety and toxicity grade parameters are listed as the following:

| Parameter | Safe level | Toxicity level |
|---|---|---|
| White blood cell count | 4.3-10.8 × 109 /L | >100,000/uL |
| Red blood cell count | 4.15-4.9 × 1012/L | |
| Platelet count | 130-400 × 109/L | <25,000/uL |
| Hematocrit | M: 0.42-0.52 | |
| | F: 0.37-0.48 | |
| Hemoglobin | | |
| plasma | 0.01-0.05 g/L | |
| whole blood | M: 8.1-11.2 mmol/L | |
| | F: 7.4-9.9 mmol/L | |
| Albumin | 35-55 g/L | |
| Glucose | <7.8 mmol/L | Impaired: 7.8-11.1 |
| | | mmol/L |
| | | Diabetes: >11.1 |
| | | mmol/L |
| Cholesterol | <200 mg/dL | >240 mg/dL |
| Urea Nitrogen | 3.6-7.1 mg/dL | |
| Aminotransferase | | |
| Aspartate (AST) | 0-0.58 ukat/L | |
| Alanine (ALT) | 0-0.58 ukat/L | |

4. The Immune Response

Aside from the immune response of Fetuin taking into account parameters such as surface IgG, lymphocyte, B-cells, Thy1.2 antigen and antibody forming cells, we also observed the antigenic response after Fetuin injection, and no hypersensitivity reaction was shown. However, the species are mice, rat and rabbits to bovine, not human to bovine. The amino acid sequence of Fetuin homologue between species is about 65%. Because the mechanism of Fetuin exerting the drug effect is by binding to the growth factors, this is expected to have less immune response than other protein nature drugs.

A. Selection of Patients

Eligibility Criteria

a. Histologic or cytologic diagnosis of cancer. Both solid tumors and hematologic malignancies (lymphoma, leukemia, multiple myeloma) will be included.

b. Age 18 years of age or older.

c. All patients' tumors must be refractory to known forms of effective therapy as well as other investigational agents of higher potential efficacy.

d. A Karnofsky performance status >50% and a predicted life expectancy of at least 12 weeks.

e. Patients must have been off anticancer therapy for at least 3 weeks (six weeks if a prior nitrosoure or mitomycin-C) and have recovered from the toxic effects of that treatment.

f. Adequate hematologic lab values: ANC 0.1000/mm3, platelets >75,000/mm3, hemoglobin>8.0 gm/dL. Growth factor therapy and transfusions are allowed. For patients with leukemias, these criteria will be waived.

g. Serum chemistries: bilirubin<2.0×ULN, creatinine<1.5 ULN.

h. Patients must give informed written consent prior to study participation.

i. Since the effects of Fetuin on the unborn fetus are unknown, no women who are pregnant will be entered on the study. Women of child-bearing potential must have a negative pregnant test and be using a barrier method of birth control. Men must also use a birth control method.

B. Treatment Plan

General:

This protocol will investigate an intravenous dosing schedule of Fetuin given over 1 hour, three times weekly. One cycle will be defined as a 4 weeks of treatment followed by 2 wks rest for a total duration of 6 wks per cycle.

Starting Dose:

10 mg/kg body weight. For example, a patient of 60 kg body weight at a dose of 10 mg/kg, the infusion of 100 ml contains 6 ml of Apogen F 0.1 g/ml saline and 94 ml clinical grade saline within one hour (The infusion rate is 1.67 ml/min.) At a dose of 50 mg/kg for a 60 kg patient, the infusion fluid contains 30 ml Apogen F 0.1 g/ml and 70 ml clinical grade saline in one hour infusion.

Study Design:

Three patients with cancer who are previously untreated with Fetuin will be entered at each dose level. Before the enrollment of two additional patients, the first patient entered at each level will be observed for 1 wk. Then two additional patients will be entered. At least 3 evaluable patients will be studied at each dose level, and dose escalation will not be carried out until a minimum of three evaluable patients have been assessed one week after the first dose of the first course of therapy. Following the administration of Fetuin, patients will be followed weekly. If the patients tumor is stable or shows a decrease in size, the patient will be started on a new course of Fetuin. The drug will be discontinued when any of the criteria for removal from the study are noted. The initial dose escalation scheme is as the following:

| Level | Dose | Route | Schedule | #Patients |
|---|---|---|---|---|
| 1 | 10 mg/kg BW | IV | TIW (MWF) | 3 |
| 2 | 20 mg/kg BW | IV | TIW (MWF) | 3 |
| 3 | 30 mg/kg BW | IV | TIW (MWF) | 3 |
| 4 | 40 mg/kg BW | IV | TIW (MWF) | 3 |
| 5 | 50 mg/kg BW | IV | TIW (MWF) | 3 |

Rules for Dose Escalation:

Dosage will be escalated in successive cohorts of 3 new patients so long as no unacceptable toxicity is observed. Unacceptable toxicity is defined as grade 3 nonhematologic toxicity according to NCI common toxicity criteria version 2.0. If a patient experiences an unacceptable toxicity in the first course among 3 patients, then 3 additional patients will be entered at the dose level. If fewer than 3 patients experience an unacceptable toxicity, the dose escalating will continue. If at least 50% (3/6) of patients experience an unacceptable toxicity during the first course, then the previous dose level will be declared as the maximum tolerated dose (MTD). If two or more patients among the first three have unacceptable toxicities on the first course, the investigator and sponsor will jointly decide if additional patients will be added at the dose level or the previous dose level. Patients experiencing an unacceptable toxicity may be continued on therapy for subsequent dose and courses but at the previous dose level. Dose reductions will be permanent. Patients with a grade 2 or greater allergic reaction will be removed from the study permanently.

Duration of Therapy:

Each treatment cycle will last for 4 weeks. Repeat cycles of therapy will be permitted if no dose limiting toxicity is seen in prior cohort. Patients can remain on therapy as long as stable disease or better is observed and no unacceptable toxicities are encountered.

Pharmacokinetics:

Pharmacokinetics will be attempted on at least 2 of three patients at each dose level on the first administration of the first course and the last administration of the course (day 1, day 26). An assay for Fetuin in the stored specimen will be performed after a methodology has been established. Once the dose level appear to be the MTD, at least 4 patients will undergo pharmacokinetic sampling. All pharmacokinetic samples should be accompanied by the following information: Time sample was drawn, time that the Fetuin dose was given and any concurrent medications taken on that day. Subjects will have 7 ml of blood in a green top tube drawn at the following times: Day 1—0 hr (immediately prior to IV infusion), at end of infusion, 5 min. post infusion, 15 min ., 30 min., 1 hr., 1.5 hr., 2 hr., 4 hr., 8 hr., and 24 hr post infusion.

C. Toxicity Management

Definition of Dose Limiting Toxicity (DLT):

This study will use the cancer therapy evaluation program common toxicity criteria (CTC) version 2.0 for toxicity and adverse event reporting. DLT is defined as any Grade 3 or 4 toxicity attributed to study drug administration, according to the CTC version 2.0 with the following exception:

a. Lymphopenia is not dose-limiting regardless of grade.

b. Grade 3 Leukopenia, Neutropenia, Thrombocytopenia, or Anemia are not dose limiting.

The following Grade 2 toxicity is dose limiting: Allergic reaction.

Dose Adjustments for DLT:

Dose adjustment may be necessary due to toxicity. The investigator must use the available data and exercise clinical judgement in deciding whether or not an adverse event is study related. The decision regarding treatment adjustment or discontinuation from study drug therapy for suspected drug associated toxicity must follow the protocol guidelines but ultimately rests with the investigator. If a DLT is observed, then the following guidelines for dose modifications should be adhered to:

a. With the first occurrence of a DLT: Hold the study medication until the DLT resolves <grade 1. Once the DLT resolves, the study medication should then be resumed at the next lowest dose level. If the subject was already on the lowest level, the medication should be resumed at half the starting dose.

b. Recurrent DLT: There will be no limit on the number of de-escalations a patient must have provided that the toxicity resolves to <grade 1.

c. Classification of Adverse Events by Severity and Relationship to Study Drug Administration.

D. Study Parameters and Calendar

| | Pre- | | Weekly | Prior to | End of |
|---|---|---|---|---|---|
| Parameter | treatment | Cycle 1 | (days)1 | each cycle* | study |
| History & Physical Exam | x | | x | x | x |
| Weight, Performance Status | x | | x | x | x |
| Toxicity Assessment2 | | | x | x | x |
| WBC (differential) ,Hgb,platelets | x | | x | x | x |
| Electrolytes, BUN, Cr | x | | x | x | x |
| LFT (SGOT and total bili) | x | | x | x | x |
| Urinalysis | x | | | x | x |
| PT/PTT | x | | | x | x |
| EKG | x | | | x3 | |
| CXR | x | | | x4 | |
| Tumor biopsy | x5 | | | | |
| Radiology, X-ray or scans for | x | | | x6 | x |
| disease measurement6 | | | | | |
| Blood for pharmacokinetics7 | x | | | x | |

*Each cycle is 42 days.
1Increase to 3 times weekly if grade 4 neutropenia occurs on day 7 or 14.
2Use NCI common toxicity criteria
3As clinically indicated
4If used to follow measurable disease
5If feasible
6If measurable disease is present. To be performed after completion of treatment period at cycle 2 and or end of study
7To be done in ancillary pharmacology protocol when active

Disease Status

a. Measurable disease: Bidimensionable measurable lesions with clearly defined margins by 1) medical photograph (skin or oral lesions) or Plain X-ray, with at least one diameter 0.5 cm or greater (bone lesions not included) or 2) CT, MRI, or other imaging scan, with both diameters greater than the distance between cuts of the imaging study or 30 palpation, with both diameters 2 cm or greater.

b. Evaluable disease: Unidimensionally measurable lesions, masses with margins not clearly defined, lesions with both diameters less than 0.5 cm, lesions on scan with either diameter smaller than the distance between cuts, palpable lesions with either diameter less than 2 cm, bone disease (markers which have been shown to be highly correlated with extent of disease are also considered to be evaluable).

c. Nonevaluable disease: Pleural effusions, ascites, disease documented by indirect evidence only (e.g. by lab values).

Use of the definition is illustrated in next table with several sequences of objective status and the corresponding best response.

Special Instruction

Pharmacokinetics:

Fetuin assay will be performed by ELISA and by HPLC. Total plasma samples (EDTA tubes) will be collected and frozen at −70° C. on first and last infusions of cycle 1. Drug levels will be determined on at least 2 patients on each dose level.

### II. Prostate Cancer

In case of clinical protocol for prostate cancer, we use the similar design but particularly addressing to prostate cancer symptoms and response. A critical component of cancer management is assessing the response to treatment. In addition to a careful examination in which all sites of disease are physically measured and recorded in a flow chart by date, response assessment usually requires periodic repeating of imaging tests that are abnormal at the time of staging. If imaging tests have become normal, repeat biopsy of previously involved tissue is performed to document completely response by pathology criteria. The most common prostate cancer screening modalities are digital rectal examination and assays for serum prostate-specific antigen (PSA), a profound prostate cancer tumor marker. The increase and decrease in PSA level usually manifest the tumor burden in patient management. The measurement of free and bound PSA in serum and urine will be a parameter to determine the effectiveness of cancer treatment beside the whole panel of hematological evaluation, clinical chemistry and histopathological analysis. The observation of anorexia, weight loss and jaundice to recurrent and progressive tumor during treatment or after treatment. The exact dose will be determined by body weight, age and the analysis of transforming growth factors, cytokines and hormones representing in the designed differential display in individual patients. The number of treatment will be determined by the parameters of patient examination. The route of injection is suggested through the middle of tumor or subcutaneous. The trace of patient history after treatment every 1, 3 and 6 months will be used to assess the side effect of the treatment. The starting dose is suggested to be 10 mg/kg body weight and escalating as proposed in the above section.

The dose schedule, duration and route of administration is proposed as the following:

General:

This protocol will investigate an subcutaneous dosing of Fetuin at daily schedule for 2 weeks. One cycle will be defined as a 2 wks of treatment followed by 2 wks rest for a total duration of 4 wks per cycle.

Starting Dose:

10 mg/kg body weight

Study Design:

Three patients with cancer who are previously untreated with Fetuin will be entered at each dose level. Before the enrollment of two additional patients, the first patient entered at each level will be observed for 1 wk. Then two additional patients will be entered. At least 3 evaluable patients will be studied at each dose level, and dose escalation will not be carried out until a minimum of three evaluable patients have been assessed one week after the first dose of the first course of therapy. Following the administration of Fetuin, patients will be followed weekly. If the patients tumor is stable or shows a decrease in size, the patient will be started on a new course of Fetuin. The drug will be discontinued when any of the criteria for removal from the study are noted. The initial dose escalation scheme is as the following:

| Level | Dose | Route | Schedule | #Patients |
|---|---|---|---|---|
| 1 | 10 mg/kg BW | SC | MTWTF | 3 |
| 2 | 20 mg/kg BW | SC | MTWTF | 3 |
| 3 | 30 mg/kg BW | SC | MTWTF | 3 |
| 4 | 40 mg/kg BW | SC | MTWTF | 3 |
| 5 | 50 mg/kg BW | SC | MTWTF | 3 |

Duration of Therapy:

Each treatment cycle will last for 4 weeks. Repeat cycles of therapy will be permitted if no dose limiting toxicity is seen in prior cohort. Patients can remain on therapy as long as stable disease or better is observed and no unacceptable toxicities are encountered.

Pharmacokinetics:

Pharmacokinetics will be attempted on at least 2 of three patients at each dose level on the first administration of the first course and the last administration of the course (day 1, day 14). An assay for Fetuin in the stored specimen will be performed after a methodology has been established. Once the dose level appear to be the MTD, at least 4 patients will undergo pharmacokinetic sampling. All pharmacokinetic samples should be accompanied by the following information: Time sample was drawn, time that the Fetuin dose was given and any concurrent medications taken on that day. Subjects will have 7 ml of blood in a green top tube drawn at the following times: Day 1—0 hr (immediately prior to IP injection), 5 min. post injection, 15 min ., 30 min., 1 hr., 1.5 hr., 2 hr., 4 hr., 8 hr., and 24 hr post injection.

### III. Anti-Aging Medicine

Fetuin is a secretory protein in extracellular fluid that will transport growth factors to target tissues by binding to growth factors and cytokines. It plays a role of regulatory factor for cell growth, differentiation and apoptosis during embryogenesis. Apoptosis is an important biological function for cell regeneration during growing stage. High concentration of Fetuin will induce apoptosis and stimulate metabolism and regeneration of new cell population. In other words, Fetuin can keep the cell population in a young stage. Low concentration of Fetuin just keep the cell in a growth arrest stage and lead to cell senescence. For decades, scientists linked cell senescence to an enzyme, telomerase which will shorten the chromosome end of each cell division cycle. Premature aging study linked to an enzyme, 3′-5′ exonuclease. In fact, it is the enzyme deleted an important gene, Fetuin that regulates cell growth and apoptosis extracellularly. Genomic study indicated that telomerase RNA gene is located at chromosome 3q26.3, very close adjacent to another gene, Fetuin (at chromosome 3q27) that is directly linked to growth arrest and apoptosis. However, contraindications exist to directly correlate telomerase and tumorgenesis as follows: (i) Telomerase RNA expression can be detected in absence of enzyme activity. (ii) The tumor incidence that connect to telomerase activity only at chromosome 3 but not the other chromosomes. (iii) The expression of hTERT catalytic unit linked to tumor incidences only at stage I and II but not stage III. The shortening of stage III is in fact a deletion of Fetuin gene that directly affect the cell growth and apoptosis that related to the cell senescence and tumorgenesis. Therefore, the biological phenomenon of telomerase we observed is indeed a biological function of Fetuin.

Many malignant patients were found either to have reduced level of Fetuin or an absence of Fetuin depending on the extent of chromosome 3 shortening. Based on our theory, many questions need to address the role of Fetuin in cell senescence and aging-related disease such as tumorgenesis. Previous data in our laboratory indicated fetal Fetuin can induce apoptosis in cancer cells but not the normal cells. In addition, the apoptosis inducing activity of Fetuin is dependent upon the carbohydrate modification and the quantity of Fetuin. Fetal Fetuin was found to be sialic acid-rich (˜8.7%) and mature Fetuin was sialic acid-poor (˜0.2%). More interestingly, fetal Fetuin can induce apoptosis, however, mature Fetuin lose the apoptosis-inducing activity.

Several senescence mechanisms had been proposed such as (1) Caloric restriction delayed the aging process that is related to mitochondria deficiency for glucose metabolism, glycosylation and reactive oxygen species. There are 100 proteins involved in the oxidative stress and a quarter of them identified involved in carbohydrate metabolism for glycosylation. Since Fetuin is such an important glycoprotein in our body, the major protein in mitochondria processing and the glycosylation of Fetuin molecule affect the apoptosis-inducing activity that regulate the regeneration of cell population during aging, we need to elucidate the correlation between Fetuin and aging-related metabolism. (2) Gene silence as a regulator for life span: Beside glucose, there are other energy utilization such as ATP and Nicotinamide Adenine Dinucleotide (NAD) synthesis and silence information regulator for cell cycle. Senescence related to the hyper-repression of gene, especially a gene that directly related to growth arrest and apoptosis as Fetuin during aging process. (3) Hormonal control of aging: Aging is a programmed cell death controlled by endocrine system such as hormone. Research in C. elegans life span indicated that a single gene mutation in Insulin-like Growth Factor (IGF-1) extend the life span significantly. The IGF-1 also regulate an alternate developmental state called Dauer. Dauer is a growth-arrested, stress-resistant alternate larval stage that is induced by food limitation and crowding. The dauer state can be considered as a puberty checkpoint, as it arrests growth just prior to reproductive maturation. Only young larvae can become dauers; once the animals have entered “puberty”, they no longer have this option. In mammal, thyroid hormone control the metabolic process of growth and maturation of tissues, cell respiration and total energy expenditure, and turnover of essential all substrate, vitamins and hormones. Two classes of thyroid hormone receptor (TR), alpha and beta, are encoded by genes on chromosome 17 and 3, respectively. The TR protein are part of a superfamily of genes encoded by mRNAs of cellular homologues of the avian retroviral v-erb A protooncogene. The TR genes, c-erb-A-alpha and c-erb-A-beta encode receptor proteins with a T3-binding domain and a DNA-binding domain. The DNA-binding domain attaches to regulatory sequences in those target genes with T3-regulated transcription [e.g. Thyroid-Stimulating Hormone (TSH), prolactin and growth hormone genes.] Fetuin is a molecule that is related to this regulatory function. Hormone regulated the expression of a regulatory factor that directly control growth arrest and apoptosis, Fetuin. Many cytokines and growth factors down-regulate the expression of Fetuin. The fact that the carbohydrate property of Fetuin changed the apoptosis-inducing activity indicated that the synthesis of Fetuin is programmed and developmentally regulated, in other words as differential glycosylation. Fetuin is an important molecule that related to the whole process of embryogenesis, growing stage, aging and tumorgenesis.

Whether the property of Fetuin varies at different ages is unknown. We need to address how this is related to the aging-related diseases, especially cancer. The fact that neuramindase, an enzyme that hydrolyze the terminal carbohydrate, sialic acid, is a marker of tumor cells and neuramindase abolished the apoptosis-inducing activity of Fetuin, which lead to tumor cell outgrowth, strongly support the notion that Fetuin can be applied in treatment of anti-aging medical research. It is essential to understand the role of Fetuin in cell senescence and aging-related diseases since it is a carrier protein for many important biological function and growth.

Cell senescence is a process of reduced regeneration of normal cell population, i.e. less apoptosis due to reduced level of Fetuin or in absence of Fetuin. The supplement of Fetuin will revive the cell population in a young, active condition. Several parameters that determine the aging process will be examined under Fetuin treatment. Since hormone was found to activate the telomerase, we have reason to believe the expression of Fetuin is developmentally regulated by hormone and is closely related to the whole process of cell growth and aging.

The most frequently occurred aging-related disease is cancer. The etiology of cancer was believed as the followings: (1) Virus infection resulted in the over expression of some genes that is related to growth factors named oncogenes such as erb, src and ras. (2) Chemical mutation in the diet or environment that lead to the carcinogenesis. (3) Genetic damages passed through generation from generation that may be expressed or suppressed. This may be due to the silence information regulator as the second senescence mechanism illustrated above. (4) Aging-related tumorgenesis, in this case, is the deletion of an important gene that regulate the regeneration of normal cell population and the outgrowth of tumor cell population. No matter what mechanism it is, it is all related to the expression of Fetuin and the property of Fetuin which directly control the cell growth and apoptosis. This is the main purpose of our study. Further, we can extend our study in the effect of Fetuin for accelerated senescence model.

Previous data in our laboratory indicated that the carbohydrate modification of Fetuin may determine the tertiary structure of Fetuin whether it will expose the binding domain of growth factor that regulate the biological function. We have strong evidences to believe the biological phenomenon of telomerase is the shortening of Fetuin gene in the aging process. A differential display study in a Switzerland group indicated that Liver Cirrhosis is caused by a defective Fetuin expression, a deleted Fetuin was found as a 41 kDa glycoprotein instead of full length as 48 kDa precursor and its expression level is reduced to 45% of normal, strongly support our theory.(24) Furthermore, in a premature aging study named Werner Syndrome with all the disorders as aging such as atherosclerosis, cancer, type 2 diabetes, osteoporosis, cataracts, wrinkled skin and grey hair, has been linked to a protein as 3′-5′ exonuclease.(25) Since Fetuin is such an important molecule in our body affecting many profound function of many tissues, whether the defect of Fetuin during aging is in the DNA level, RNA levels or protein modification levels in mitochondria processing is useful in diagnosis and therapy. This can be achieved by analysis of DNA, RNA transcript and protein functional activity as proposed in this project.

The significance of this study is to apply our theory in diagnosis, prevention and therapy of aging-related diseases by analysis of carbohydrate property of AHSG that is due to the deletion of AHSG gene during aging, and most importantly to find the perfect timing for Fetuin treatment to prevent the aging syndrome. We claim the usage of Fetuin as an anti-aging medicine.

Experimental Design and Methods

1. Fetuin Molecule Analysis at Different Ages

Fetuin expression is varied during embryogenesis quantitatively and qualitatively during mature stage. Since it is a molecule that its expression is developmentally regulated, it is essential to analyze the property of Fetuin at different age and interpret the results to the regeneration ability of our cell population during aging.

A. Extraction of Fetuin from serum samples of different age of patients

The following protocol was used for Fetuin analysis:

0.5 ml serum add 0.4 ml H2O, 0.3 ml 0.1 M Zn(Ac)2 and 0.3 ml 95% EtOH dry ice 5 min., spin at microcentrifuge for 3 min.

Transfer 0.8 ml supernatant, add 0.2 ml 1M Ba(Ac)2 and 0.3 ml 95% EtOH dry ice 5 min., spin at microcentrifuge for 3 min.

Transfer 1 ml supernatant, add 0.3 ml 95% EtOH, incubate O/N, spin at microcentrifuge for 3 min.

Pellet suspended in 100 ul H2O, spin 3 min at microcentrifuge to remove residues.

Sample hydrolyzed in 0.025 N Sulphuric acid at 80 0 C for 1 hr.

Sialic acid was analyzed with HPLC quantitatively.

B. The glycoprotein will be analyzed by 2-D gel electrophoresis reported by Nicolle Packer et al. Several glycoisoforms will be determined by Dionex DX 500 carbohydrate system and recorded ESI spectra using Quattro II triple quadrupole MS.

C. The tertiary structure of Fetuin will send out to subcontractor to be determined respectively and compared to the sialic acid content. The apoptosis-inducing activity of Fetuin purified from different ages and the binding capacity to TGF will be evaluated.

2. How Hormone Affect the Expression of Fetuin That is Related to Age

Since fetal and mature Fetuin have different carbohydrate characteristics that related to growth regulation, we believe the property of Fetuin is related to hormone secretion and age. In this study, we would like to study how different hormones treatment affect the expression of Fetuin, in terms of glycosylation modification and quantity. We analyze the property of Fetuin after growth hormone (GH) treatment in secretory gland model or animal model.

A. In secretory gland tissue model we analyze the Fetuin property in conditioned media and in animal model we analyze the Fetuin property in serum as suggested in the above section.

B. One liter of conditioned media from secretory gland tissue treated with GH and 10 ml of serum collected from animal injected with GH were purified homogeneously according to the above protocol for Fetuin analysis.

C. Different ages of tissue and different ages of animal were planned to use in the study to observe the property of Fetuin.

3. Analysis of Fetuin for Aging-Related Diseases

Since many malignant patients were found to have reduced level of Fetuin or in absence of Fetuin, we would like to study whether the defect of patients is in the DNA level, RNA transcript level or glycosylation modification level by analysis of Fetuin in different aging-related disease patients serum.

A. How Fetuin property or defect linked to clinical study of patients with aging-related diseases.

B. Differential display study of Fetuin in various cancer patients.

C. The study of Fetuin in a premature aging model or accelerated senescence model.

4. Fetuin Therapy for Aging-Related Diseases

We already assessed the efficacy and safety of Fetuin treatment in species such as mice, rats and rabbits and would like to apply Fetuin treatment in patients with symptom of Fetuin defect for therapy of patients in above section.

A. Clinical assessment of Fetuin treatment in patients with Fetuin defect related to aging diseases.

B. Diagnosis application of Fetuin analysis in clinical cases.

C. Prevention in reversible senescence study.

5. Alpha 2-HS Glycoprotein (AHSG) Expression at Transcription Level During Pathogenesis

Recent molecular study on AHSG indicated that the potent promoter region at 5′-transcription control is accounted for by C/EBP and NF-1 binding site.

Many of the outlines depended on the results to plan. However, this is a whole picture of our goal and points we would like to achieve in the study. We present the results of preclinical study of Fetuin treatment in the previous section to support the proposal of our study. This study can proceed at the same time of section II by collecting blood sample of patients and used in different approach. The main concern is whether deletion of Fetuin linked to aging-related diseases and to find the perfect timing for Fetuin therapy.

Fetuin play a regulatory role in controlling embryogenesis, growing stage and aging. It is the key molecule for proliferation and apoptotic function of pattern formation, cell regeneration and defective at senescence lead to tumorgenesis and neuro-degenerated diseases. There are three applications as followed: (1) Neurologic disorder at childhood and neuro-degenerated disease at aging. (2) Anti-cancer therapy: Tumorgenesis is a matter of growth control. Whether onset of cancer due to virus infection, environmental factors or diet, it can be controlled by an extracellular regulatory factor to compensate the defective growth control mechanism although it has different affinity in specific tissues. Many malignant patients were found to have either in absence or reduced level of Fetuin. A differential display system to facilitate diagnosis and accurate therapy dose is planed following the study. (3) Anti-aging medicine: Since deletion of Fetuin is linked to telomerase activity, cell senescence and tumorgenesis, it is important to elucidate the perfect timing of Fetuin treatment. I has to emphasize the potency of Fetuin regulation is more efficient than any other therapy because it is directly targeted on growth factors and cytokines which is the upstream of growth control. In conclusion, it is a natural regulatory factor with specificity and selectivity.

In another embodiment, there is an alternative in the small molecule approach in therapeutic use. This involves the setting of a high-throughput screening assay searching for small molecule targeting on the repression or inhibitor of Telomerase. This is related to the auto-immune deficiency of AHSG due to aging.

### IV. Therapy of Acquired Immune Deficiency (AID)

The invention also relates to the usage of AHSG and its related clones in therapy of AIDS due to the its regulatory role in T-cell and B-cell proliferative or suppressive modulation. Interferons are cytokines that exhibit a wide spectrum of antiviral activities as well as immunomodulating and antiproliferative properties. The loss of immunity is due to the failure to compensate for lost T-cell homeostatic mechanism. In HIV-infected individuals, potentially reversible suppression is exerted at a very early stage of T cell lymphopoiesis, presumably at the level of thymocytes or thymocyte precursors.

The thymus has been shown to be a site for HIV replication, and products of HIV replication may potentially exert a reversible suppressive effect on maturing thymocytes. Activation of CD8 T cells driven by HIV replication leads to excessive production of cytokines, which may suppress T lymphocyte precursors in the bone marrow or thymus. After some time the suppressive effect becomes irreversible, causing permanent thymic dysfunction and failure to generate new CD4 T cells in place of the CD4 T cells lost, an event that is known to precede the onset of clinical immunodeficiency. The tolerance for such suppression is reduced with age due to T cell degeneration and shortening of telomerase resulting defective AHSG gene.

AHSG can bind sarcolectin, cytokines such as IL-4 and reduce the production of TNF-alpha in macrophage activation present in many therapeutic properties in curing immunodeficiency-related diseases. The expression of AHSG is down-regulated by growth factors and cytokines. The binding of AHSG to transforming growth factor-beta is linked to the onset of anti-cancer immunity. The binding characteristics of AHSG present an antagonized effect in cytokine production and TGF activation. Fetuin, human homologue, can induce apoptosis of HL-60 cells at 1 uM. An increasing 5-fold dose and treated of healthy animals did not affect the red blood cell count, white blood cell count and platelet count.

Fetuin treatment also did not affect the antigen-presenting cells and create any immunosuppression. This suggested that the apoptosis-inducing activity of AHSG is specific and selective in the infected cell population but does not affect the normal cell population. All these qualities of AHSG may produce a drug without any side-effects in therapy of AIDS.

### V. Therapy of Bone-Related Diseases

The invention also relates to the usage of AHSG and its related clones in therapy of bone-related diseases such as Paget's disease, Rheumatoid Arthritis, Osteoporosis and Osteoarthritis which occur during degeneration. AHSG has sequence homology to Bone Resorption Protein (BRP) and plays a role in osteogenesis as calcium phosphate absorbent for osteoblast mineralization. In other words, AHSG can bind Bone Morphogenic Protein and its expression is programmed to be extremely high, about 30-100 fold higher than normal cells and induce apoptosis for bone formation. The onset of many bone-related diseases including cancellous bone was found to have clinical signs of reduced levels of AHSG and high TGF-beta. It is also associated with the glycosylation forms of AHSG. AHSG at a young age is highly glycosylated with an unusual negatively-charged terminal carbohydrate, named sialic acid. The negative charge of sialic acid increases the calcium-binding capacity. The glycosylation form is controlled by growth hormone that regulates the metabolic rate of carbohydrate. The over expression of androgen hormone often results in clinical signs of Extramammary Paget disease. AHSG also has sequence homology to Leukemia inhibitory factor and differentiation-stimulating factor. Its regulatory role is dependent on two factors: (1) Relative concentration to growth factors and cytokines; and (2) Glycosylation form that determines the tertiary structure of the molecule whether it exposes the binding site of active domain. When cell population is degenerated, there are two possibilities: (1) Metabolic turnover rate is reduced thus resulting in the synthesis of mature AHSG which apoptosis activity for cell regeneration is abolished. (2) Due to telomerase activity, a certain population of AHSG gene is deleted that loses the antagonized domain and cell growth leads to a cancellous stage. The therapy of AHSG can control the aggressive growth of cancer cells to an extent and even induce apoptosis of cancer cells.

AHSG also can reduce TNF-alpha production and bind IL-4. Endochondral ossification begins from the condensation and differentiation of mesenchymal cells into cartilage. The cartilage then goes through a programmed proliferation, hypertrophic differentiation, calcification, apoptosis, and is even replaced by bone. Unlike most cartilage, articular cartilage is arrested before hypertrophic differentiation. The onset of inflammatory tissues is dependent on the repressing articular chondrocyte differentiation. Inhibition of chondrocytes break the quiescent state and undergo abnormal terminal differentiation ultimately leading to osteoarthritis. The pathogenic basis of these diseases was dependent on the over expression of TGF-beta during virus transformation. Since AHSG is able to bind TGF-beta and antagonized the proliferatively inflammatory site due to virus infection, the supplement of AHSG can compensate the defected change due to Neuramindase activity of bacteria and virus, or degenerated function of cells due to aging, particularly related to bone diseases.

### VI. Differential Display in Diagnosis

In mammalian cells, antimitogenic signal cause growth arrest in the G₁ phase of the cell division cycle. Differentiation and senescence are two common cellular processes associated with cell cycle withdrawal by antimitogenic factors. The cellular process initiated by the cell cycle block depend on the nature of the extracellular signals. Genomic study linked the AHSG gene function to growth arrest and apoptosis. AHSG is the extracellular molecule that controls the cell cycle process by relative binding to growth factors. High concentration of AHSG will growth arrest permanently in G₀phase and lead to senescence. Cyclin D-cdk 4/6 is accumulated highest in G₀ phase and finally induce apoptosis of the cells. However, when malignancy occurred, the progression or transition from G₁ to S phase is rapid. It leads to accumulation of cyclin E-cdk2 expression. Therefore, cyclin D and B can be the markers in diagnosis.

Virus infection often leads to the overexpression of oncogenic proteins in transcriptional levels. The development of a kit of differential display on some related diagnosis markers will facilitate the accurate treatment of patients. The nuclear extract of tissue can use probes of diagnosis markers to check the change of expression levels due to virus infections. Cancer derived from the abnormal antagonized function related to AHSG including hypocalcemia and hyperparathyroidism (a multi-endocrine neoplasia such as tumors with pancreas and pituitary origin, often associated with hormone hypersecretion that affect the metabolic rate), can have manifestation of TSH expression which will down-regulate the expression of AHSG. AEV is a retro virus which induces both erythroleukemia and fibrosarcoma. The v-erb B oncogene derived from a host cell gene for the epidermal growth factor receptor, whereas the v-erb A oncogene, in contrast, is not itself sufficient for oncogenic transformation but instead acts in neoplasia by blocking differentiation of infected erythroid cells and by altering the growth requirement of infected fibroblasts. The v-erb A oncogene is derived from c-erb A-alpha, a gene for a thyroid hormone receptor. They act as hormone-regulated transcription factors. All members of nuclear hormone receptor family share a common modular structure consisting of a cysteine-rich “Zinc-finger” domain that confers DNA binding and a COOH-terminal domain that binds the hormone ligand. AHSG is the cysteine family transcription factor that antagonized the growth and is regulated by hormone, especially growth hormone, prolactin and thyroid.

Neuroblastoma is a highly malignant pediatric tumor derived from the neural crest. Neuritic extension is the differentiation stage that related to delayed cyclin D inhibition and cell cycle arrest, in the absence of Ink4 activity but with high p21 Cip/p27 Kip activity. The increase in p21^{CIPI} takes place in the presence of abundant cyclin D1-cdk4/cdk6 complexes. The p21^{Cip1} expression induced significant neurite extension and the extracellular molecule that controls the cell cycle process is the relative concentration of AHSG and neurotrophic factor. One significant symptom of malignancy would be the overexpression of cyclin E which leads to abnormal cell replication or cyclin D which leads to growth arrest or senescence. This characteristic offers an ideal diagnosis marker in solid tumor tissues.

Based on this evidence, a differential display kit is designed to cover all important diagnosis parameters related to AHSG and used for cancer therapy. The parameters are composed of transforming growth factor-beta, AHSG itself, TSH and cyclin D/E. Cyclin D/E can provide information about the diagnosis of cancer stage. TGF and AHSG will facilitate the drug administration of dose calculation. TSH is the clinical symptom of abnormal endocrine secretion that is related to AHSG. In summary, this differential display kit is the by-product of AHSG-related diagnosis and therapy.

In addition to cyclins' regulation, the expression of various isoforms and hormones modulate the transcriptional events that lead to various biological functions. The specificity is relied on DNA-binding affinity as reporter gene, hormone binding activity as modulation and steroid and thyroid initiator as immunogenic antagonization. AHSG molecules possess all these binding domains and are responsible for various functions. Therefore, AHSG clones have unique function and can be used in therapy in many aspects of diseases. The linking biology of various isoforms of AHSG explained the symptoms of various tissue dysfunction or deficiency. The target of drug effect is very specific and presents less side-effect than other therapies. For example, EEC syndrome is an autosomal dominant disorder characterized by ectrodactyly, ectodermal dysplasia and facial clefts. The genetic defect has been mapped to chromosome 3q27, p63 gene. Eight mutations results in amino acid substitution in DNA-binding region of p63 and abolished the transcription. The ninth is a frameshift mutation that affects p63 alpha but not p63 beta and p63 gamma isotypes. The importance of p63alpha is obvious in deleted mutation study. AHSG clones can compensate for genetic defects that present abnormal deficiency in fundamental roles. Another example would be Parkinson's disease, which is the translocation of 3q27-29 to chromosome 19, affecting the linking transcription of associated protein. Chronic Myelogenous Leukemia is linked to p63 mutation in DNA-binding domain p63 is expressed in the basal cells of many epithelial organs and its germline inactivation in the mouse results in agenesis of organs such as skin appendages and the breast. p63 protein is not detected in human prostate adenocarcinoma. Deletion of p63 affect the prostate development. An interaction of p63 alpha protein with SV40 large tumor antigen was detected and ectopic expression of DeltaNp63 can extend the life-span of rat embryo fibroblasts. p63 plays a role in replicative senescence either by competition for p53 DNA binding sites or by interaction with p53 bound to DNA. In normal tissues, isoform TAp63 is present in much higher amount than DeltaNp63 whereas in the bladder carcinoma tissue, reduced expression of TAp63 is correlated to tumor stage and grade. In summary, p63 is responsible for various organ function by hormone modulation and is a reporter gene as DNA-binding for transcription. Importantly, it served as an auto-immunity agent induced by IL. With profound multi-role functions, AHSG clones can fix many genetic defects in fundamental cell growth, differentiation and apoptosis as a regulatory factor which can be shown in differential display studies. The supplement of various AHSG clones can compensate the defective expression of p63 in various cases quantitatively or qualitatively.

1. A method of inducing apoptosis in cancer cells by administering alpha 2-HS glycoproteins (AHSG) to said cancer cells.

2. A method of inducing apoptosis in cancer cells by administering alpha 2-HS glycoproteins (AHSG) to said cancer cells, wherein said cancer cells are selected from the group consisting of prostate cancer cell, leukemia cells, breast cancer cells, colon cancer cells or lung cancer cells.

3. A method a treating neurologic disorders by inducing apoptosis of neuroblastoma, the method comprising administering effective amounts alpha 2-HS glycoproteins (AHSG) to cells having neuroblastoma presence.

4. A method as claimed in claim 3 wherein the neurologic disorders treated are selected form the group consisting of learning disability, paralysis, memory loss, eye sight deficiency, hearing deficiency, and seizure.

5. A method as claimed in claim 1 wherein the cancer cells are prostate cancer cells.

6. A method as claimed in claim 5 wherein the alpha 2-HS glycoprotein are dosed at about 10 mg/kg body weight.

7. A method for treating cells to replicative senescence, the method comprising the administering of Fetuin to the cells.

8. A method for treating acquired immuno deficiency syndrome (AIDS) by inducing apoptosis in HIV-infected cell populations, the method comprising the step of administering fetuin to said cell populations.

9. A method for treating acquired immuno deficiency syndrome (AIDS) by inducing apoptosis in HIV-infected cell populations, the method comprising the step of administering alpha 2-HS glycoproteins (AHSG) or a related clone thereof to said cell populations.

10. A method for treating bone diseases by inducing apoptosis in bone cell populations, the method comprising the step of administering alpha 2-HS glycoproteins (AHSG) or a related clone thereof to said cell populations.

11. A method for treating bone diseases by inducing apoptosis in bone cell populations, the method comprising the step of administering fetuin to said cell populations.

12. A method as claimed in claim 10 wherein glycosylation forms of alpha 2-HS glycoproteins (AHSG) are used.

13. A method as claimed in claim 9 wherein the bone diseases treated are selected from the group consisting of Paget's disease, Rheumatoid arthritis, Osteoporosis and Osteoarthritis.
